(19) United States
(12) Patent Application Publication
(10) Pub. No.: US 2014/0178944 A1
(43) Pub. Date: Jun. 26, 2014

(54) Preservation of Biomass for Pretreatment

(71) Applicant: Sweetwater Energy, Inc., Rochester, NY (US)

(72) Inventors: Sarad Parekh, Pittsford, NY (US); Carl P. Felice, Churchville, NY (US)

(21) Appl. No.: 13/931,303

(22) Filed: Jun. 28, 2013

Related U.S. Application Data
(60) Provisional application No. 61/666,777, filed on Jun. 29, 2012.

(51) Int. Cl. C12P 19/04

(52) U.S. Cl. CPC C12P 19/04
USPC 435/99; 435/101

(57) ABSTRACT

Provided are methods and compositions directed to increasing the rate and/or yield of sugar extraction processes using feedstocks. Also provided are methods and compositions for decreasing the yield of one or more undesirable products during pretreatment.

### CROSS-REFERENCE

This application claims the benefit of U.S. Provisional Application No. 61/666,777, filed Jun. 29, 2012 which is incorporated herein by reference in its entirety.

### BACKGROUND OF THE INVENTION

The ability to convert lignocellulosic feedstocks into fuels and chemicals has advantages over the use of fossil fuels, including the advantages of the ready availability of large amounts and varieties of biomass, the avoidance of burning or land filling with waste materials, and the relatively easy conversion of carbonaceous polymers into useful products. Although corn ethanol plants are the primary source of renewable energy fuels at present, substantial progress has been made of conversion techniques that reduce the cost of using other plant and organic waste sources of energy-rich biomass.

One challenge is to reduce the costs of pretreating the biomass in the process of extracting the sugars contained therein. Another is to ensure the sources of feedstocks year round without the loss of valuable polymers. Thus, storage of biomass that is harvested and held many months until processing is initiated can be crucial. If not properly preserved, spoilage can result in a reduction of total sugars from the stored materials. This is especially true if harvested material has too high a moisture content or is stored under normal atmospheric conditions. Ensiling biomass is one method to preserve the integrity of the chemical composition. However, this often requires the addition of bacterial inoculants, enzymes or inhibitors which can alter the composition and/or which require washing of the biomass prior to pretreatment. Even with the addition of special agents, proper environmental conditions for preservation can be lost. Therefore, improved techniques are needed to preserve the biochemical composition of a biomass following harvesting.

### SUMMARY OF THE INVENTION

Disclosed herein are methods of producing silage from a biomass, the methods comprising: packing the biomass in a container to a bulk density sufficient to reduce oxygen levels within and surrounding the biomass; contacting the biomass with sulfur dioxide gas, formic acid, propionic acid, or a combination thereof to acidify the biomass; and maintaining the biomass under conditions and for a time to produce silage from the biomass. Also disclosed herein are methods of producing one or more sugar streams from a biomass, the methods comprising: packing the biomass in a container to a bulk density sufficient to reduce oxygen levels within and surrounding the biomass; contacting the biomass with sulfur dioxide gas, formic acid, propionic acid, or a combination thereof to acidify the biomass; maintaining the biomass under conditions and for a time to produce silage from the biomass; and hydrolyzing the silage to produce one or more sugar streams. In some embodiments, the silage does not require washing prior to a pretreatment. In some embodiments, less lactic acid is produced compared to an ensiling process that does not include contacting a biomass with sulfur dioxide gas, formic acid, propionic acid, or a combination thereof. In some embodiments, no lactic acid is produced. Some embodiments further comprise pretreating the silage by dilute acid hydrolysis, wherein less acid is required during pretreatment than for silage produced by an ensiling process that does not include contacting a biomass with sulfur dioxide gas, formic acid, propionic acid, or a combination thereof. In some embodiments, the one or more sugar streams comprise a higher yield of monomeric sugars in comparison to a yield from silage produced by an ensiling process that does not include contacting a biomass with sulfur dioxide gas, formic acid, propionic acid, or a combination thereof. In some embodiments, the silage contains a lower level of bacteria, mold, and/or yeast than a silage produced by an ensiling process that does not include contacting a biomass with sulfur dioxide gas, formic acid, propionic acid, or a combination thereof. In some embodiments, the biomass is acidified to a pH of from about 4 to about 6. In some embodiments, the formic acid is in an amount of from about 0.1% to about 20% of the biomass by dry matter weight. In some embodiments, the formic acid is in an amount of from about 1% to about 5% of the biomass by dry matter weight. In some embodiments, the sulfur dioxide is in an amount of from about 0.1% to about 20% of the biomass by dry matter weight. In some embodiments, the sulfur dioxide is in an amount of from about 1% to about 5% of the biomass by dry matter weight. In some embodiments, the propionic acid is in an amount of from about 0.1% to about 20% of the biomass by wet matter weight. In some embodiments, the propionic acid is in an amount of from about 0.1% to about 5% of the biomass by wet matter weight. Some embodiments further comprise contacting the biomass with carbon dioxide to reduce oxygen levels. In some embodiments, the biomass is reduced in size prior to packing the biomass in the container. In some embodiments, the biomass is reduced to an average size of from about 0.6 to about 1.3 cm prior to packing the biomass in the container. In some embodiments, the bulk density is from about 10 lbs/ft³ to about 100 lbs/ft³. In some embodiments, the bulk density is from about 20 lbs/ft³ to about 60 lbs/ft³. In some embodiments, the biomass is reduced in size prior to packing the biomass in the container. In some embodiments, the biomass is reduced to an average size of from about 0.6 to about 1.3 cm prior to packing the biomass in the container. In some embodiments, the biomass has a moisture content of from about 50% to about 70%. In some embodiments, the silage is stored in a container. In some embodiments, the silage is stored in an air-tight container. In some embodiments, the silage comprises non-cellulosic sugars. In some embodiments, the silage comprises non-cellulosic oligosaccharides. In some embodiments, the silage comprises starch. Some embodiments further comprise contacting the biomass with an amylase. In some embodiments, the biomass comprises crops or crop residues from grasses, herbaceous legumes, tree legumes, corns or maize, alfalfas, sorghums or sweet sorghums, oats, rice, wheat, barley, millets, triticale, ryes, buckwheat, fonios, quinoa, or a combination thereof. In some embodiments, the biomass comprises corn, corn syrup, corn stover, corn cobs, molasses, grass, straw, grain hulls, bagasse, distiller's grains, distiller's dried solubles, distiller's dried grains, condensed distiller's solubles, distiller's wet grains, distiller's dried grains with solubles, wood, bark, sawdust, paper, poplars, willows, switchgrass, alfalfa, prairie bluestem, algae, fruit peels, pits, sorghum, sweet sorghum, sugar cane, switch grass, rice, rice straw, rice hulls, wheat, wheat straw, barley, barley straw, bamboo, seeds, seed hulls, oats, oat hulls, food waste, municipal sewage waste, or a combination thereof.

Also disclosed herein are methods of producing silage from a biomass that does not require prewashing for pretreatment comprising: packing the biomass to a bulk density sufficient to reduce oxygen levels within and surrounding the biomass; contacting the biomass with SO₂ gas to reduce pH; and maintaining the biomass under low oxygen and low pH for sufficient time to produce silage from the biomass. In some embodiments, the biomass is reduced in size prior to the packing In some embodiments, the biomass is reduced to an average size of from about 0.6 cm to about 1.3 cm. In some embodiments, the biomass is reduced in size by chopping, cutting, shredding, chipping, grinding, hammer-milling, or a combination thereof. In some embodiments, the bulk density is from about 10 lbs/ft³ to about 100 lbs/ft³. In some embodiments, the bulk density is from about 20 lbs/ft³ to about 60 lbs/ft³. In some embodiments, the silage is stored in a container. In some embodiments, the container is an air-tight container. In some embodiments, the silage pH is maintained between pH 4 to pH 6. In some embodiments, the silage is used to produce fermentation end-products. In some embodiments, the silage comprises non-cellulosic sugars. In some embodiments, the silage comprises non-cellulosic oligosaccharides. In some embodiments, the silage comprises starch. In some embodiments, the silage is also treated with amylase during the storage process or prior to pretreatment. In some embodiments, one or more monosaccharides are produced from the pretreatment and/or hydrolysis of cellulose, hemicellulose, or lignocellulose material in the silage. In some embodiments, the silage comprises corn, corn syrup, corn stover, corn cobs, molasses, grass, straw, grain hulls, bagasse, distiller's grains, distiller's dried solubles, distiller's dried grains, condensed distiller's solubles, distiller's wet grains, distiller's dried grains with solubles, wood, bark, sawdust, paper, poplars, willows, switchgrass, alfalfa, prairie bluestem, algae, fruit peels, pits, sorghum, sweet sorghum, sugar cane, switch grass, rice, rice straw, rice hulls, wheat, wheat straw, barley, barley straw, bamboo, seeds, seed hulls, oats, oat hulls, food waste, municipal sewage waste, or a combination thereof.

Also disclosed herein are processes of making silage for pretreatment which comprises intimately mixing with moisture-containing chopped vegetation from which the silage is produced a quantity of formic acid, and storing the resultant mixture in a reduced oxygen atmosphere, the amount of formic acid being sufficient to enhance the acidity of the silage so it can be stored without significant yeast or mold fermentation. In some embodiments, said chopped vegetation is a forage crop comprising one or more fermentable feedstocks selected from the group consisting of corn syrup, molasses, silage, agricultural residues, including genetically-modified or in other ways improved agricultural plants, seed hulls, corn cobs, corn, sorghum, switchgrass, bagasse, animal waste, Distillers Dried Solubles (DDS), Distillers Dried Grains (DDG), Condensed Distillers Solubles (CDS), Distillers Wet Grains (DWG), Distillers Dried Grains with Solubles (DDGS), woody materials, municipal waste, and energy crops. In some embodiments, the moisture containing biomass contains at least 40% by weight water. In some embodiments, the quantity of formic acid is within the range of 0.15% to 0.65% of the wet matter weight of the biomass. In some embodiments, the quantity of formic acid is within the range of 0.75% to 4.25% of the dry matter weight of the biomass. In some embodiments, SO₂ gas is substituted for formic acid and the quantity of SO₂ gas is within the range of 0.5% to 5.0% of the dry matter weight of the biomass In some embodiments, SO₂ gas is substituted for formic acid and the quantity of SO₂ gas is added to the dry matter weight of the biomass until the overall pH is between 3.0 to 6.0. In some embodiments, propionic acid or CO₂ is substituted for formic acid and is used to reduce oxygen and/or lower acidity.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

As used in the specification and the appended claims, the singular forms “a,” “an” and “the” include plural referents unless the context clearly dictates otherwise. Thus, for example, reference to “a purified monomer” includes mixtures of two or more purified monomers. The term “comprising” as used herein is synonymous with “including,” “containing,” or “characterized by,” and is inclusive or open-ended and does not exclude additional, unrecited elements or method steps.

“About” means a referenced numeric indication plus or minus 10% of that referenced numeric indication. For example, the term about 4 would include a range of 3.6 to 4.4. All numbers expressing quantities of ingredients, reaction conditions, and so forth used in the specification are to be understood as being modified in all instances by the term “about.” Accordingly, unless indicated to the contrary, the numerical parameters set forth herein are approximations that can vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Wherever the phrase “for example,” “such as,” “including” and the like are used herein, the phrase “and without limitation” is understood to follow unless explicitly stated otherwise. Therefore, “for example ethanol production” means “for example and without limitation ethanol production.”

In this specification and in the claims that follow, reference will be made to a number of terms that shall be defined to have the following meanings

### DEFINITIONS

“Optional” or “optionally” means that the subsequently described event or circumstance may or may not occur, and that the description includes instances where said event or circumstance occurs and instances where it does not. For example, the phrase “the medium can optionally contain glucose” means that the medium may or may not contain glucose as an ingredient and that the description includes both media containing glucose and media not containing glucose.

Unless characterized otherwise, technical and scientific terms used herein have the same meaning as commonly understood by one of ordinary skill in the art.

“Fermentive end-product” and “fermentation end-product” are used interchangeably herein to include biofuels, chemicals, compounds suitable as liquid fuels, gaseous fuels, triacylglycerols (TAGs), reagents, chemical feedstocks, chemical additives, processing aids, food additives, bioplastiks and precursors to bioplastiks, and other products. Examples of fermentive end-products include but are not limited to 1,4 diacids (succinic, fumaric and malic), 2,5 furan dicarboxylic acid, 3 hydroxy propionic acid, aspartic acid, glucaric acid, glutamic acid, itaconic acid, levulinic acid, 3-hydroxybutyrolactone, glycerol, sorbitol, xylitol/arabinitol, butanediol, butanol, methane, methanol, ethane, ethene, ethanol, n-propane, 1-propene, 1-propanol, propanal, acetone, propionate, n-butane, 1-butene, 1-butanol, butanal, butanoate, isobutanal, isobutanol, 2-methylbutanal, 2-methylbutanol, 3-methylbutanal, 3-methylbutanol, 2-butene, 2-butanol, 2-butanone, 2,3-butanediol, 3-hydroxy-2-butanone, 2,3-butanedione, ethylbenzene, ethenylbenzene, 2-phenylethanol, phenylacetaldehyde, 1-phenylbutane, 4-phenyl-1-butene, 4-phenyl-2-butene, 1-phenyl-2-butene, 1-phenyl-2-butanol, 4-phenyl-2-butanol, 1-phenyl-2-butanone, 4-phenyl-2-butanone, 1-phenyl-2,3-butandiol, 1-phenyl-3-hydroxy-2-butanone, 4-phenyl-3-hydroxy-2-butanone, 1-phenyl-2,3-butanedione, n-pentane, ethylphenol, ethenylphenol, 2-(4-hydroxyphenyl)ethanol, 4-hydroxyphenylacetaldehyde, 1-(4-hydroxyphenyl) butane, 4-(4-hydroxyphenyl)-1-butene, 4-(4-hydroxyphenyl)-2-butene, 1-(4-hydroxyphenyl)-1-butene, 1-(4-hydroxyphenyl)-2-butanol, 4-(4-hydroxyphenyl)-2-butanol, 1-(4-hydroxyphenyl)-2-butanone, 4-(4-hydroxyphenyl)-2-butanone, 1-(4-hydroxyphenyl)-2,3-butandiol, 1-(4-hydroxyphenyl)-3-hydroxy-2-butanone, 4-(4-hydroxyphenyl)-3-hydroxy-2-butanone, 1-(4-hydroxyphenyl)-2,3-butanonedione, indolylethane, indolylethene, 2-(indole-3-)ethanol, n-pentane, 1-pentene, 1-pentanol, pentanal, pentanoate, 2-pentene, 2-pentanol, 3-pentanol, 2-pentanone, 3-pentanone, 4-methylpentanal, 4-methylpentanol, 2,3-pentanediol, 2-hydroxy-3-pentanone, 3-hydroxy-2-pentanone, 2,3-pentanedione, 2-methylpentane, 4-methyl-1-pentene, 4-methyl-2-pentene, 4-methyl-3-pentene, 4-methyl-2-pentanol, 2-methyl-3-pentanol, 4-methyl-2-pentanone, 2-methyl-3-pentanone, 4-methyl-2,3-pentanediol, 4-methyl-2-hydroxy-3-pentanone, 4-methyl-3-hydroxy-2-pentanone, 4-methyl-2,3-pentanedione, 1-phenylpentane, 1-phenyl-1-pentene, 1-phenyl-2-pentene, 1-phenyl-3-pentene, 1-phenyl-2-pentanol, 1-phenyl-3-pentanol, 1-phenyl-2-pentanone, 1-phenyl-3-pentanone, 1-phenyl-2,3-pentanediol, 1-phenyl-2-hydroxy-3-pentanone, 1-phenyl-3-hydroxy-2-pentanone, 1-phenyl-2,3-pentanedione, 4-methyl-1-phenylpentane, 4-methyl-1-phenyl-1-pentene, 4-methyl-1-phenyl-2-pentene, 4-methyl-1-phenyl-3-pentene, 4-methyl-1-phenyl-3-pentanol, 4-methyl-1-phenyl-2-pentanol, 4-methyl-1-phenyl-3-pentanone, 4-methyl-1-phenyl-2-pentanone, 4-methyl-1-phenyl-2,3-pentanediol, 4-methyl-1-phenyl-2,3-pentanedione, 4-methyl-1-phenyl-3-hydroxy-2-pentanone, 4-methyl-1-phenyl-2-hydroxy-3-pentanone, 1-(4-hydroxyphenyl) pentane, 1-(4-hydroxyphenyl)-1-pentene, 1-(4-hydroxyphenyl)-2-pentene, 1-(4-hydroxyphenyl)-3-pentene, 1-(4-hydroxyphenyl)-2-pentanol, 1-(4-hydroxyphenyl)-3-pentanol, 1-(4-hydroxyphenyl)-2-pentanone, 1-(4-hydroxyphenyl)-3-pentanone, 1-(4-hydroxyphenyl)-2,3-pentanediol, 1-(4-hydroxyphenyl)-2-hydroxy-3-pentanone, 1-(4-hydroxyphenyl)-3-hydroxy-2-pentanone, 1-(4-hydroxyphenyl)-2,3-pentanedione, 4-methyl-1-(4-hydroxyphenyl) pentane, 4-methyl-1-(4-hydroxyphenyl)-2-pentene, 4-methyl-1-(4-hydroxyphenyl)-3-pentene, 4-methyl-1-(4-hydroxyphenyl)-1-pentene, 4-methyl-1-(4-hydroxyphenyl)-3-pentanol, 4-methyl-1-(4-hydroxyphenyl)-2-pentanol, 4-methyl-1-(4-hydroxyphenyl)-3-pentanone, 4-methyl-1-(4-hydroxyphenyl)-2-pentanone, 4-methyl-1-(4-hydroxyphenyl)-2,3-pentanediol, 4-methyl-1-(4-hydroxyphenyl)-2,3-pentanedione, 4-methyl-1-(4-hydroxyphenyl)-3-hydroxy-2-pentanone, 4-methyl-1-(4-hydroxyphenyl)-2-hydroxy-3-pentanone, 1-indole-3-pentane, 1-(indole-3)-1-pentene, 1-(indole-3)-2-pentene, 1-(indole-3)-3-pentene, 1-(indole-3)-2-pentanol, 1-(indole-3)-3-pentanol, 1-(indole-3)-2-pentanone, 1-(indole-3)-3-pentanone, 1-(indole-3)-2,3-pentanediol, 1-(indole-3)-2-hydroxy-3-pentanone, 1-(indole-3)-3-hydroxy-2-pentanone, 1-(indole-3)-2,3-pentanedione, 4-methyl-1-(indole-3-)pentane, 4-methyl-1-(indole-3)-2-pentene, 4-methyl-1-(indole-3)-3-pentene, 4-methyl-1-(indole-3)-1-pentene, 4-methyl-2-(indole-3)-3-pentanol, 4-methyl-1-(indole-3)-2-pentanol, 4-methyl-1-(indole-3)-3-pentanone, 4-methyl-1-(indole-3)-2-pentanone, 4-methyl-1-(indole-3)-2,3-pentanediol, 4-methyl-1-(indole-3)-2,3-pentanedione, 4-methyl-1-(indole-3)-3-hydroxy-2-pentanone, 4-methyl-1-(indole-3)-2-hydroxy-3-pentanone, n-hexane, 1-hexene, 1-hexanol, hexanal, hexanoate, 2-hexene, 3-hexene, 2-hexanol, 3-hexanol, 2-hexanone, 3-hexanone, 2,3-hexanediol, 2,3-hexanedione, 3,4-hexanediol, 3,4-hexanedione, 2-hydroxy-3-hexanone, 3-hydroxy-2-hexanone, 3-hydroxy-4-hexanone, 4-hydroxy-3-hexanone, 2-methylhexane, 3-methylhexane, 2-methyl-2-hexene, 2-methyl-3-hexene, 5-methyl-1-hexene, 5-methyl-2-hexene, 4-methyl-1-hexene, 4-methyl-2-hexene, 3-methyl-3-hexene, 3-methyl-2-hexene, 3-methyl-1-hexene, 2-methyl-3-hexanol, 5-methyl-2-hexanol, 5-methyl-3-hexanol, 2-methyl-3-hexanone, 5-methyl-2-hexanone, 5-methyl-3-hexanone, 2-methyl-3,4-hexanediol, 2-methyl-3,4-hexanedione, 5-methyl-2,3-hexanediol, 5-methyl-2,3-hexanedione, 4-methyl-2,3-hexanediol, 4-methyl-2,3-hexanedione, 2-methyl-3-hydroxy-4-hexanone, 2-methyl-4-hydroxy-3-hexanone, 5-methyl-2-hydroxy-3-hexanone, 5-methyl-3-hydroxy-2-hexanone, 4-methyl-2-hydroxy-3-hexanone, 4-methyl-3-hydroxy-2-hexanone, 2,5-dimethylhexane, 2,5-dimethyl-2-hexene, 2,5-dimethyl-3-hexene, 2,5-dimethyl-3-hexanol, 2,5-dimethyl-3-hexanone, 2,5-dimethyl-3,4-hexanediol, 2,5-dimethyl-3,4-hexanedione, 2,5-dimethyl-3-hydroxy-4-hexanone, 5-methyl-1-phenylhexane, 4-methyl-1-phenylhexane, 5-methyl-1-phenyl-1-hexene, 5-methyl-1-phenyl-2-hexene, 5-methyl-1-phenyl-3-hexene, 4-methyl-1-phenyl-1-hexene, 4-methyl-1-phenyl-2-hexene, 4-methyl-1-phenyl-3-hexene, 5-methyl-1-phenyl-2-hexanol, 5-methyl-1-phenyl-3-hexanol, 4-methyl-1-phenyl-2-hexanol, 4-methyl-1-phenyl-3-hexanol, 5-methyl-1-phenyl-2-hexanone, 5-methyl-1-phenyl-3-hexanone, 4-methyl-1-phenyl-2-hexanone, 4-methyl-1-phenyl-3-hexanone, 5-methyl-1-phenyl-2,3-hexanediol, 4-methyl-1-phenyl-2,3-hexanediol, 5-methyl-1-phenyl-3-hydroxy-2-hexanone, 5-methyl-1-phenyl-2-hydroxy-3-hexanone, 4-methyl-1-phenyl-3-hydroxy-2-hexanone, 4-methyl-1-phenyl-2-hydroxy-3-hexanone, 5-methyl-1-phenyl-2,3-hexanedione, 4-methyl-1-phenyl-2,3-hexanedione, 4-methyl-1-(4-hydroxyphenyl)hexane, 5-methyl-1-(4-hydroxyphenyl)-1-hexene, 5-methyl-1-(4-hydroxyphenyl)-2-hexene, 5-methyl-1-(4-hydroxyphenyl)-3-hexene, 4-methyl-1-(4-hydroxyphenyl)-1-hexene, 4-methyl-1-(4-hydroxyphenyl)-2-hexene, 4-methyl-1-(4-hydroxyphenyl)-3-hexene, 5-methyl-1-(4-hydroxyphenyl)-2-hexanol, 5-methyl-1-(4-hydroxyphenyl)-3-hexanol, 4-methyl-1-(4-hydroxyphenyl)-2-hexanol, 4-methyl-1-(4-hydroxyphenyl)-3-hexanol, 5-methyl-1-(4-hydroxyphenyl)-2-hexanone, 5-methyl-1-(4-hydroxyphenyl)-3-hexanone, 4-methyl-1-(4-hydroxyphenyl)-2-hexanone, 4-methyl-1-(4-hydroxyphenyl)-3-hexanone, 5-methyl-1-(4-hydroxyphenyl)-2,3-hexanediol, 4-methyl-1-(4-hydroxyphenyl)-2,3-hexanediol, 5-methyl-1-(4-hydroxyphenyl)-3-hydroxy-2-hexanone, 5-methyl-1-(4-hydroxyphenyl)-2-hydroxy-3-hexanone, 4-methyl-1-(4-hydroxyphenyl)-3-hydroxy-2-hexanone, 4-methyl-1-(4-hydroxyphenyl)-2-hydroxy-3-hexanone, 5-methyl-1-(4-hydroxyphenyl)-2,3-hexanedione, 4-methyl-1-(4-hydroxyphenyl)-2,3-hexanedione, 4-methyl-1-(indole-3-)hexane, 5-methyl-1-(indole-3)-1-hexene, 5-methyl-1-(indole-3)-2-hexene, 5-methyl-1-(indole-3)-3-hexene, 4-methyl-1-(indole-3)-1-hexene, 4-methyl-1-(indole-3)-2-hexene, 4-methyl-1-(indole-3)-3-hexene, 5-methyl-1-(indole-3)-2-hexanol, 5-methyl-1-(indole-3)-3-hexanol, 4-methyl-1-(indole-3)-2-hexanol, 4-methyl-1-(indole-3)-3-hexanol, 5-methyl-1-(indole-3)-2-hexanone, 5-methyl-1-(indole-3)-3-hexanone, 4-methyl-1-(indole-3)-2-hexanone, 4-methyl-1-(indole-3)-3-hexanone, 5-methyl-1-(indole-3)-2,3-hexanediol, 4-methyl-1-(indole-3)-2,3-hexanediol, 5-methyl-1-(indole-3)-3-hydroxy-2-hexanone, 5-methyl-1-(indole-3)-2-hydroxy-3-hexanone, 4-methyl-1-(indole-3)-3-hydroxy-2-hexanone, 4-methyl-1-(indole-3)-2-hydroxy-3-hexanone, 5-methyl-1-(indole-3)-2,3-hexanedione, 4-methyl-1-(indole-3)-2,3-hexanedione, n-heptane, 1-heptene, 1-heptanol, heptanal, heptanoate, 2-heptene, 3-heptene, 2-heptanol, 3-heptanol, 4-heptanol, 2-heptanone, 3-heptanone, 4-heptanone, 2,3-heptanediol, 2,3-heptanedione, 3,4-heptanediol, 3,4-heptanedione, 2-hydroxy-3-heptanone, 3-hydroxy-2-heptanone, 3-hydroxy-4-heptanone, 4-hydroxy-3-heptanone, 2-methylheptane, 3-methylheptane, 6-methyl-2-heptene, 6-methyl-3-heptene, 2-methyl-3-heptene, 2-methyl-2-heptene, 5-methyl-2-heptene, 5-methyl-3-heptene, 3-methyl-3-heptene, 2-methyl-3-heptanol, 2-methyl-4-heptanol, 6-methyl-3-heptanol, 5-methyl-3-heptanol, 3-methyl-4-heptanol, 2-methyl-3-heptanone, 2-methyl-4-heptanone, 6-methyl-3-heptanone, 5-methyl-3-heptanone, 3-methyl-4-heptanone, 2-methyl-3,4-heptanediol, 2-methyl-3,4-heptanedione, 6-methyl-3,4-heptanediol, 6-methyl-3,4-heptanedione, 5-methyl-3,4-heptanediol, 5-methyl-3,4-heptanedione, 2-methyl-3-hydroxy-4-heptanone, 2-methyl-4-hydroxy-3-heptanone, 6-methyl-3-hydroxy-4-heptanone, 6-methyl-4-hydroxy-3-heptanone, 5-methyl-3-hydroxy-4-heptanone, 5-methyl-4-hydroxy-3-heptanone, 2,6-dimethylheptane, 2,5-dimethylheptane, 2,6-dimethyl-2-heptene, 2,6-dimethyl-3-heptene, 2,5-dimethyl-2-heptene, 2,5-dimethyl-3-heptene, 3,6-dimethyl-3-heptene, 2,6-dimethyl-3-heptanol, 2,6-dimethyl-4-heptanol, 2,5-dimethyl-3-heptanol, 2,5-dimethyl-4-heptanol, 2,6-dimethyl-3,4-heptanediol, 2,6-dimethyl-3,4-heptanedione, 2,5-dimethyl-3,4-heptanediol, 2,5-dimethyl-3,4-heptanedione, 2,6-dimethyl-3-hydroxy-4-heptanone, 2,6-dimethyl-4-hydroxy-3-heptanone, 2,5-dimethyl-3-hydroxy-4-heptanone, 2,5-dimethyl-4-hydroxy-3-heptanone, n-octane, 1-octene, 2-octene, 1-octanol, octanal, octanoate, 3-octene, 4-octene, 4-octanol, 4-octanone, 4,5-octanediol, 4,5-octanedione, 4-hydroxy-5-octanone, 2-methyloctane, 2-methyl-3-octene, 2-methyl-4-octene, 7-methyl-3-octene, 3-methyl-3-octene, 3-methyl-4-octene, 6-methyl-3-octene, 2-methyl-4-octanol, 7-methyl-4-octanol, 3-methyl-4-octanol, 6-methyl-4-octanol, 2-methyl-4-octanone, 7-methyl-4-octanone, 3-methyl-4-octanone, 6-methyl-4-octanone, 2-methyl-4,5-octanediol, 2-methyl-4,5-octanedione, 3-methyl-4,5-octanediol, 3-methyl-4,5-octanedione, 2-methyl-4-hydroxy-5-octanone, 2-methyl-5-hydroxy-4-octanone, 3-methyl-4-hydroxy-5-octanone, 3-methyl-5-hydroxy-4-octanone, 2,7-dimethyloctane, 2,7-dimethyl-3-octene, 2,7-dimethyl-4-octene, 2,7-dimethyl-4-octanol, 2,7-dimethyl-4-octanone, 2,7-dimethyl-4,5-octanediol, 2,7-dimethyl-4,5-octanedione, 2,7-dimethyl-4-hydroxy-5-octanone, 2,6-dimethyloctane, 2,6-dimethyl-3-octene, 2,6-dimethyl-4-octene, 3,7-dimethyl-3-octene, 2,6-dimethyl-4-octanol, 3,7-dimethyl-4-octanol, 2,6-dimethyl-4-octanone, 3,7-dimethyl-4-octanone, 2,6-dimethyl-4,5-octanediol, 2,6-dimethyl-4,5-octanedione, 2,6-dimethyl-4-hydroxy-5-octanone, 2,6-dimethyl-5-hydroxy-4-octanone, 3,6-dimethyloctane, 3,6-dimethyl-3-octene, 3,6-dimethyl-4-octene, 3,6-dimethyl-4-octanol, 3,6-dimethyl-4-octanone, 3,6-dimethyl-4,5-octanediol, 3,6-dimethyl-4,5-octanedione, 3,6-dimethyl-4-hydroxy-5-octanone, n-nonane, 1-nonene, 1-nonanol, nonanal, nonanoate, 2-methylnonane, 2-methyl-4-nonene, 2-methyl-5-nonene, 8-methyl-4-nonene, 2-methyl-5-nonanol, 8-methyl-4-nonanol, 2-methyl-5-nonanone, 8-methyl-4-nonanone, 8-methyl-4,5-nonanediol, 8-methyl-4,5-nonanedione, 8-methyl-4-hydroxy-5-nonanone, 8-methyl-5-hydroxy-4-nonanone, 2,8-dimethylnonane, 2,8-dimethyl-3-nonene, 2,8-dimethyl-4-nonene, 2,8-dimethyl-5-nonene, 2,8-dimethyl-4-nonanol, 2,8-dimethyl-5-nonanol, 2,8-dimethyl-4-nonanone, 2,8-dimethyl-5-nonanone, 2,8-dimethyl-4,5-nonanediol, 2,8-dimethyl-4,5-nonanedione, 2,8-dimethyl-4-hydroxy-5-nonanone, 2,8-dimethyl-5-hydroxy-4-nonanone, 2,7-dimethylnonane, 3,8-dimethyl-3-nonene, 3,8-dimethyl-4-nonene, 3,8-dimethyl-5-nonene, 3,8-dimethyl-4-nonanol, 3,8-dimethyl-5-nonanol, 3,8-dimethyl-4-nonanone, 3,8-dimethyl-5-nonanone, 3,8-dimethyl-4,5-nonanediol, 3,8-dimethyl-4,5-nonanedione, 3,8-dimethyl-4-hydroxy-5-nonanone, 3,8-dimethyl-5-hydroxy-4-nonanone, n-decane, 1-decene, 1-decanol, decanoate, 2,9-dimethyldecane, 2,9-dimethyl-3-decene, 2,9-dimethyl-4-decene, 2,9-dimethyl-5-decanol, 2,9-dimethyl-5-decanone, 2,9-dimethyl-5,6-decanediol, 2,9-dimethyl-6-hydroxy-5-decanone, 2,9-dimethyl-5,6-decanedionen-undecane, 1-undecene, 1-undecanol, undecanal. undecanoate, n-dodecane, 1-dodecene, 1-dodecanol, dodecanal, dodecanoate, n-dodecane, 1-decadecene, n-tridecane, 1-tridecene, 1-tridecanol, tridecanal, tridecanoate, n-tetradecane, 1-tetradecene, 1-tetradecanol, tetradecanal, tetradecanoate, n-pentadecane, 1-pentadecene, 1-pentadecanol, pentadecanal, pentadecanoate, n-hexadecane, 1-hexadecene, 1-hexadecanol, hexadecanal, hexadecanoate, n-heptadecane, 1-heptadecene, 1-heptadecanol, heptadecanal, heptadecanoate, n-octadecane, 1-octadecene, 1-octadecanol, octadecanal, octadecanoate, n-nonadecane, 1-nonadecene, 1-nonadecanol, nonadecanal, nonadecanoate, eicosane, 1-eicosene, 1-eicosanol, eicosanal, eicosanoate, 3-hydroxy propanal, 1,3-propanediol, 4-hydroxybutanal, 1,4-butanediol, 3-hydroxy-2-butanone, 2,3-butandiol, 1,5-pentane diol, homocitrate, homoisocitorate, b-hydroxy adipate, glutarate, glutarsemialdehyde, glutaraldehyde, 2-hydroxy-1-cyclopentanone, 1,2-cyclopentanediol, cyclopentanone, cyclopentanol, (S)-2-acetolactate, (R)-2,3-Dihydroxy-isovalerate, 2-oxoisovalerate, isobutyryl-CoA, isobutyrate, isobutyraldehyde, 5-amino pentaldehyde, 1,10-diaminodecane, 1,10-diamino-5-decene, 1,10-diamino-5-hydroxydecane, 1,10-diamino-5-decanone, 1,10-diamino-5,6-decanediol, 1,10-diamino-6-hydroxy-5-decanone, phenylacetoaldehyde, 1,4-diphenylbutane, 1,4-diphenyl-1-butene, 1,4-diphenyl-2-butene, 1,4-diphenyl-2-butanol, 1,4-diphenyl-2-butanone, 1,4-diphenyl-2,3-butanediol, 1,4-diphenyl-3-hydroxy-2-butanone, 1-(4-hydeoxyphenyl)-4-phenylbutane, 1-(4-hydeoxyphenyl)-4-phenyl-1-butene, 1-(4-hydeoxyphenyl)-4-phenyl-2-butene, 1-(4-hydeoxyphenyl)-4-phenyl-2-butanol, 1-(4-hydeoxyphenyl)-4-phenyl-2-butanone, 1-(4-hydeoxyphenyl)-4-phenyl-2,3-butanediol, 1-(4-hydeoxyphenyl)-4-phenyl-3-hydroxy-2-butanone, 1-(indole-3)-4-phenylbutane, 1-(indole-3)-4-phenyl-1-butene, 1-(indole-3)-4-phenyl-2-butene, 1-(indole-3)-4-phenyl-2-butanol, 1-(indole-3)-4-phenyl-2-butanone, 1-(indole-3)-4-phenyl-2,3-butanediol, 1-(indole-3)-4-phenyl-3-hydroxy-2-butanone, 4-hydroxyphenylacetoaldehyde, 1,4-di(4-hydroxyphenyl)butane, 1,4-di(4-hydroxyphenyl)-1-butene, 1,4-di(4-hydroxyphenyl)-2-butene, 1,4-di(4-hydroxyphenyl)-2-butanol, 1,4-di(4-hydroxyphenyl)-2-butanone, 1,4-di(4-hydroxyphenyl)-2,3-butanediol, 1,4-di(4-hydroxyphenyl)-3-hydroxy-2-butanone, 1-(4-hydroxyphenyl)-4-(indole-3-) butane, 1-(4-hydroxyphenyl)-4-(indole-3)-1-butene, 1-di(4-hydroxyphenyl)-4-(indole-3)-2-butene, 1-(4-hydroxyphenyl)-4-(indole-3)-2-butanol, 1-(4-hydroxyphenyl)-4-(indole-3)-2-butanone, 1-(4-hydroxyphenyl)-4-(indole-3)-2,3-butanediol, 1-(4-hydroxyphenyl-4-(indole-3)-3-hydroxy-2-butanone, indole-3-acetoaldehyde, 1,4-di(indole-3-)butane, 1,4-di(indole-3)-1-butene, 1,4-di(indole-3)-2-butene, 1,4-di(indole-3)-2-butanol, 1,4-di(indole-3)-2-butanone, 1,4-di(indole-3)-2,3-butanediol, 1,4-di(indole-3)-3-hydroxy-2-butanone, succinate semialdehyde, hexane-1,8-dicarboxylic acid, 3-hexene-1,8-dicarboxylic acid, 3-hydroxy-hexane-1,8-dicarboxylic acid, 3-hexanone-1,8-dicarboxylic acid, 3,4-hexanediol-1,8-dicarboxylic acid, 4-hydroxy-3-hexanone-1,8-dicarboxylic acid, glycerol, fucoidan, iodine, chlorophyll, carotenoid, calcium, magnesium, iron, sodium, potassium, phosphate, lactic acid, acetic acid, formic acid, isoprenoids, and polyisoprenes, including rubber. Further, such products can include succinic acid, pyruvic acid, enzymes such as cellulases, polysaccharases, lipases, proteases, ligninases, and hemicellulases and may be present as a pure compound, a mixture, or an impure or diluted form.

Fermentation end-products can include polyols or sugar alcohols; for example, methanol, glycol, glycerol, erythritol, threitol, arabitol, xylitol, ribitol, mannitol, sorbitol, dulcitol, fucitol, iditol, inositol, volemitol, isomalt, maltitol, lactitol, and/or polyglycitol.

The term “fatty acid comprising material” as used herein has its ordinary meaning as known to those skilled in the art and can comprise one or more chemical compounds that include one or more fatty acid moieties as well as derivatives of these compounds and materials that comprise one or more of these compounds. Common examples of compounds that include one or more fatty acid moieties include triacylglycerides, diacylglycerides, monoacylglycerides, phospholipids, lysophospholipids, free fatty acids, fatty acid salts, soaps, fatty acid comprising amides, esters of fatty acids and monohydric alcohols, esters of fatty acids and polyhydric alcohols including glycols (e.g. ethylene glycol, propylene glycol, etc.), esters of fatty acids and polyethylene glycol, esters of fatty acids and polyethers, esters of fatty acids and polyglycol, esters of fatty acids and saccharides, esters of fatty acids with other hydroxyl-containing compounds, etc. A fatty acid comprising material can be one or more of these compounds in an isolated or purified form. It can be a material that includes one or more of these compounds that is combined or blended with other similar or different materials. It can be a material where the fatty acid comprising material occurs with or is provided with other similar or different materials, such as vegetable and animal oils; mixtures of vegetable and animal oils; vegetable and animal oil byproducts; mixtures of vegetable and animal oil byproducts; vegetable and animal wax esters; mixtures, derivatives and byproducts of vegetable and animal wax esters; seeds; processed seeds; seed byproducts; nuts; processed nuts; nut byproducts; animal matter; processed animal matter; byproducts of animal matter; corn; processed corn; corn byproducts; distiller's grains; beans; processed beans; bean byproducts; soy products; lipid containing plant, fish or animal matter; processed lipid containing plant or animal matter; byproducts of lipid containing plant, fish or animal matter; lipid containing microbial material; processed lipid containing microbial material; and byproducts of lipid containing microbial matter. Such materials can be utilized in liquid or solid forms. Solid forms include whole forms, such as cells, beans, and seeds; ground, chopped, slurried, extracted, flaked, milled, etc. The fatty acid portion of the fatty acid comprising compound can be a simple fatty acid, such as one that includes a carboxyl group attached to a substituted or un-substituted alkyl group. The substituted or unsubstituted alkyl group can be straight or branched, saturated or unsaturated. Substitutions on the alkyl group can include hydroxyls, phosphates, halogens, alkoxy, or aryl groups. The substituted or unsubstituted alkyl group can have 7 to 29 carbons and preferably 11 to 23 carbons (e.g., 8 to 30 carbons and preferably 12 to 24 carbons counting the carboxyl group) arranged in a linear chain with or without side chains and/or substitutions. Addition of the fatty acid comprising compound can be by way of adding a material comprising the fatty acid comprising compound.

The term “pH modifier” as used herein has its ordinary meaning as known to those skilled in the art and can include any material that will tend to increase, decrease or hold steady the pH of the broth or medium. A pH modifier can be an acid, a base, a buffer, or a material that reacts with other materials present to serve to raise, lower, or hold steady the pH. In one embodiment, more than one pH modifier can be used, such as more than one acid, more than one base, one or more acid with one or more bases, one or more acids with one or more buffers, one or more bases with one or more buffers, or one or more acids with one or more bases with one or more buffers. In one embodiment, a buffer can be produced in the broth or medium or separately and used as an ingredient by at least partially reacting in acid or base with a base or an acid, respectively. When more than one pH modifiers are utilized, they can be added at the same time or at different times. In one embodiment, one or more acids and one or more bases are combined, resulting in a buffer. In one embodiment, media components, such as a carbon source or a nitrogen source serve as a pH modifier; suitable media components include those with high or low pH or those with buffering capacity. Exemplary media components include acid- or base-hydrolyzed plant polysaccharides having residual acid or base, ammonia fiber explosion (AFEX) treated plant material with residual ammonia, lactic acid, corn steep solids or liquor.

“Growth phase” is used herein to describe the type of cellular growth that occurs after the “Initiation phase” and before the “Stationary phase” and the “Death phase.” The growth phase is sometimes referred to as the exponential phase or log phase or logarithmic phase.

The term “plant polysaccharide” as used herein has its ordinary meaning as known to those skilled in the art and can comprise one or more polymers of sugars and sugar derivatives as well as derivatives of sugar polymers and/or other polymeric materials that occur in plant matter. Exemplary plant polysaccharides include lignin, cellulose, starch, pectin, and hemicellulose. Others are chitin, sulfonated polysaccharides such as alginic acid, agarose, carrageenan, porphyran, furcelleran and funoran. Generally, the polysaccharide can have two or more sugar units or derivatives of sugar units. The sugar units and/or derivatives of sugar units can repeat in a regular pattern, or otherwise. The sugar units can be hexose units or pentose units, or combinations of these. The derivatives of sugar units can be sugar alcohols, sugar acids, amino sugars, etc. The polysaccharides can be linear, branched, cross-linked, or a mixture thereof. One type or class of polysaccharide can be cross-linked to another type or class of polysaccharide.

The term “saccharification” as used herein has its ordinary meaning as known to those skilled in the art and can include conversion of plant polysaccharides to lower molecular weight species that can be utilized by the organism at hand. For some organisms, this would include conversion to monosaccharides, disaccharides, trisaccharides, and oligosaccharides of up to about seven monomer units, as well as similar sized chains of sugar derivatives and combinations of sugars and sugar derivatives.

The terms “SSF” and “SHF” are known to those skilled in the art; SSF meaning simultaneous saccharification and fermentation, or the conversion from polysaccharides or oligosaccharides into monosaccharides at the same time and in the same fermentation vessel wherein monosaccharides are converted to another chemical product such as ethanol. “SHF” indicates a physical separation of the polymer hydrolysis or saccharification and fermentation processes.

The term “biomass” as used herein has its ordinary meaning as known to those skilled in the art and can include one or more biological materials that can be converted into a biofuel, chemical or other product. Biomass as used herein is synonymous with the term “feedstock” and includes corn syrup, molasses, silage, agricultural residues (corn stalks, grass, straw, grain hulls, bagasse, etc.) including genetically-modified or in other ways improved agricultural plants, animal waste (manure from cattle, poultry, and hogs), Distillers Dried Solubles (DDS), Distillers Dried Grains (DDG), Condensed Distillers Solubles (CDS), Distillers Wet Grains (DWG), Distillers Dried Grains with Solubles (DDGS), woody materials (wood or bark, sawdust, timber slash, and mill scrap), municipal waste (waste paper, recycled toilet papers, yard clippings, etc.), and energy crops (poplars, willows, switchgrass, alfalfa, prairie bluestem, algae, including macroalgae, etc.). One exemplary source of biomass is plant matter. Plant matter can be, for example, woody plant matter, non-woody plant matter, cellulosic material, lignocellulosic material, hemicellulosic material, carbohydrates, pectin, starch, inulin, fructans, glucans, corn, sugar cane, grasses, switchgrass, sorghum, high biomass sorghum, bamboo, algae and material derived from these. Plants can be in their natural state or genetically modified, e.g., to increase the cellulosic or hemicellulosic portion of the cell wall, or to produce additional exogenous or endogenous enzymes to increase the separation of cell wall components. Plant matter can be further described by reference to the chemical species present, such as proteins, polysaccharides and oils. Polysaccharides include polymers of various monosaccharides and derivatives of monosaccharides including glucose, fructose, lactose, galacturonic acid, rhamnose, etc. Plant matter also includes agricultural waste byproducts or side streams such as pomace, corn steep liquor, corn steep solids, distillers grains, peels, pits, fermentation waste, straw, lumber, sewage, garbage and food leftovers. Peels can be citrus which include, but are not limited to, tangerine peel, grapefruit peel, orange peel, tangerine peel, lime peel and lemon peel. These materials can come from farms, forestry, industrial sources, households, etc. Another non-limiting example of biomass is animal matter, including, for example milk, meat, fat, animal processing waste, and animal waste. “Feedstock” is frequently used to refer to biomass being used for a process, such as those described herein.

The term “productivity” as used herein has its ordinary meaning as known to those skilled in the art and can include the mass of a material of interest produced in a given time in a given volume. Units can be, for example, grams per liter-hour, or some other combination of mass, volume, and time. In fermentation, productivity is frequently used to characterize how fast a product can be made within a given fermentation volume. The volume can be referenced to the total volume of the fermentation vessel, the working volume of the fermentation vessel, or the actual volume of broth being fermented. The context of the phrase will indicate the meaning intended to one of skill in the art. Productivity is different from “titer” in that productivity includes a time term, and titer is analogous to concentration. Titer and Productivity can generally be measured at any time during the fermentation, such as at the beginning, the end, or at some intermediate time, with titer relating the amount of a particular material present or produced at the point in time of interest and the productivity relating the amount of a particular material produced per liter in a given amount of time. The amount of time used in the productivity determination can be from the beginning of the fermentation or from some other time, and go to the end of the fermentation, such as when no additional material is produced or when harvest occurs, or some other time as indicated by the context of the use of the term. “Overall productivity” refers to the productivity determined by utilizing the final titer and the overall fermentation time.

“Titer” refers to the amount of a particular material present in a fermentation broth. It is similar to concentration and can refer to the amount of material made by the organism in the broth from all fermentation cycles, or the amount of material made in the current fermentation cycle or over a given period of time, or the amount of material present from whatever source, such as produced by the organism or added to the broth. Frequently, the titer of soluble species will be referenced to the liquid portion of the broth, with insolubles removed, and the titer of insoluble species will be referenced to the total amount of broth with insoluble species being present, however, the titer of soluble species can be referenced to the total broth volume and the titer of insoluble species can be referenced to the liquid portion, with the context indicating the which system is used with both reference systems intended in some cases. Frequently, the value determined referenced to one system will be the same or a sufficient approximation of the value referenced to the other.

“Concentration” when referring to material in the broth generally refers to the amount of a material present from all sources, whether made by the organism or added to the broth. Concentration can refer to soluble species or insoluble species, and is referenced to either the liquid portion of the broth or the total volume of the broth, as for “titer.”

The term “biocatalyst” as used herein has its ordinary meaning as known to those skilled in the art and can include one or more enzymes and/or microorganisms, including solutions, suspensions, and mixtures of enzymes and microorganisms. In some contexts this word will refer to the possible use of either enzymes or microorganisms to serve a particular function, in other contexts the word will refer to the combined use of the two, and in other contexts the word will refer to only one of the two. The context of the phrase will indicate the meaning intended to one of skill in the art. For example, a biocatalyst can be a fermenting microorganism.

The terms “conversion efficiency” or “yield” as used herein have their ordinary meaning as known to those skilled in the art and can include the mass of product made from a mass of substrate. The term can be expressed as a percentage yield of the product from a starting mass of substrate. For the production of ethanol from glucose, the net reaction is generally accepted as:

C₆H₁₂O₆→2 C₂H₅OH+2 CO₂

and the theoretical maximum conversion efficiency, or yield, is 51% (wt.). Frequently, the conversion efficiency will be referenced to the theoretical maximum, for example, “80% of the theoretical maximum.” In the case of conversion of glucose to ethanol, this statement would indicate a conversion efficiency of 41% (wt.). The context of the phrase will indicate the substrate and product intended to one of skill in the art.

“Pretreatment” or “pretreated” is used herein to refer to any mechanical, chemical, thermal, biochemical process or combination of these processes whether in a combined step or performed sequentially, that achieves disruption or expansion of the biomass so as to render the biomass more susceptible to attack by enzymes and/or microbes. In one embodiment, pretreatment includes removal or disruption of lignin so as to make the cellulose and hemicellulose polymers in the plant biomass more available to cellulolytic enzymes and/or microbes, for example, by treatment with acid or base. In one embodiment, pretreatment includes disruption or expansion of cellulosic and/or hemicellulosic material. Steam explosion, and ammonia fiber expansion (or explosion) (AFEX) are well known thermal/chemical techniques. Hydrolysis, including methods that utilize acids, bases, and/or enzymes can be used. Other thermal, chemical, biochemical, enzymatic techniques can also be used.

“Fed-batch” or “fed-batch fermentation” is used herein to include methods of culturing microorganisms where nutrients, other medium components, or biocatalysts (including, for example, enzymes, fresh organisms, extracellular broth, genetically modified plants and/or organisms, etc.) are supplied to the fermentor during cultivation, but culture broth is not harvested from the fermentor until the end of the fermentation, although it can also include “self seeding” or “partial harvest” techniques where a portion of the fermentor volume is harvested and then fresh medium is added to the remaining broth in the fermentor, with at least a portion of the inoculum being the broth that was left in the fermentor. During a fed-batch fermentation, the broth volume can increase, at least for a period, by adding medium or nutrients to the broth while fermentation organisms are present. Suitable nutrients which can be utilized include those that are soluble, insoluble, and partially soluble, including gasses, liquids and solids. In one embodiment, a fed-batch process is referred to with a phrase such as, “fed-batch with cell augmentation.” This phrase can include an operation where nutrients and cells are added or one where cells with no substantial amount of nutrients are added. The more general phrase “fed-batch” encompasses these operations as well. The context where any of these phrases is used will indicate to one of skill in the art the techniques being considered.

“Sugar compounds” or “sugar streams” is used herein to indicate mostly monosaccharide sugars, dissolved, crystallized, evaporated, or partially dissolved, including but not limited to hexoses and pentoses; sugar alcohols; sugar acids; sugar amines; compounds containing two or more of these linked together directly or indirectly through covalent or ionic bonds; and mixtures thereof. Included within this description are disaccharides; trisaccharides; oligosaccharides; polysaccharides; and sugar chains, branched and/or linear, of any length. A sugar stream can consist of primarily or substantially C6 sugars, C5 sugars, or mixtures of both C6 and C5 sugars in varying ratios of said sugars. C6 sugars have a six-carbon molecular backbone and C5 sugars have a five-carbon molecular backbone.

“C5-rich” composition means that one or more steps have been taken to remove at least some of the C6 sugars originally in the composition. For example, a C5-rich composition can include no more than about 50% C6 sugars, nor more than about 40% C6 sugars, no more than about 30% C6 sugars, no more than about 20% C6 sugars, no more than about 10% C6 sugars, no more than about 5% C6 sugars, or it can include from about 2% to about 10% C6 sugars by weight. Likewise, a “C6-rich” composition is one in which at least some of the originally-present C5 sugars have been removed. For example, a C6-rich composition can include no more than about 50% C5 sugars, nor more than about 40% C5 sugars, no more than about 30% C5 sugars, no more than about 20% C5 sugars, no more than about 10% C5 sugars, no more than about 5% C5 sugars, or it can include from about 2% to about 10% C5 sugars by weight.

A “liquid” composition may contain solids and a “solids” composition may contain liquids. A liquid composition refers to a composition in which the material is primarily liquid, and a solids composition is one in which the material is primarily solid.

“Gentle Pretreatment” generally refers to the collection of processes upstream of hydrolysis, which result in composition that, when hydrolyzed, produces a fermentable sugar composition. The fermentable sugar composition can be used to enhance a non-cellulosic fermentation process, such as a corn mash fermentation process. In some embodiments, the gentle pretreatment process provides a fermentable sugar composition having a favorable nutrient balance (e.g. plant-derived extracted nutrients, which are part of the composition as a result of the pretreatment process) and/or an amount of toxic compounds (e.g. phenolics and sugar degradation products, organic acids and furans, which inhibit and/or inactivate the performance of enzymes and or fermentation organisms), which is limited such that the resultant fermentable sugar composition can enhance a non-cellulosic fermentation process, such as a corn mash fermentation process. For example, a gentle pretreatment is one that results in a sugar stream that is about 25% (w/v) C6 sugars or more, about 4 g/L hydroxymethyl furfural or less, about 4 g/L furfural or less, about 10 g/L acetic acid or less, about 10 g/L formic acid or less for example as measured by typical HPLC methods referred to herein. (“About X amount of a substance or less” means the same as “no more than about” and includes zero—i.e. includes the possibility that none of that substance is present in the composition.) “Gentle pretreatment” can include one or more of: pre-processing biomass to reduce size and/or create size uniformity; pretreatment itself (process for making cellulose more accessible to hydrolysis); and post-processing steps such as washing steps.

The terms “non-cellulosic” and “sugar- or starch-based” are used interchangeably and have the same meaning For example “non-cellulosic fermentation process” is used interchangeably and means the same thing as “sugar- and starch-based fermentation process.” Starch is a carbohydrate consisting of consisting of a large number of glucose units joined by glycosidic bonds. The glycosidic bonds are typically the easily hydrolysable alpha glycosidic bonds. This polysaccharide can be produced by all green plants as an energy store. There can be two types of starch molecules: the linear and helical amylose and the branched amylopectin, although amylase can also contain branches.

### DESCRIPTION

The following description and examples illustrate some exemplary embodiments of the disclosure in detail. Those of skill in the art will recognize that there are numerous variations and modifications of this disclosure that are encompassed by its scope. Accordingly, the description of a certain exemplary embodiment, should not be deemed to limit the scope of the present disclosure.

Feedstock and Storage of Feedstock

The term feedstock can be used to refer to a biomass that is being used in a process, such as an ensiling process, a pretreatment process, a hydrolysis process, a fermentation process, or a combination thereof. In one embodiment, the biomass comprises cellulosic, hemicellulosic, and/or lignocellulosic material. In another embodiment, the biomass comprises, or further comprises, non-cellulosic sugars (e.g., starches). The biomass can be derived from one or more agricultural crops, crop residues, trees, woodchips, sawdust, paper, cardboard, grasses, algae, municipal waste and/or other sources.

The availability of biomass can be limited by the growing season. Methods and compositions for the storage, preservation, and/or transportation of biomass for downstream processes can be useful, for example, to enable biomass processing to occur beyond the growing season. Methods and compositions for the storage, preservation, and or transportation of biomass can enable the productions of saccharification and/or fermentation end-products from the biomass throughout the year. The methods and compositions used in the storage, preservation, and/or transportation of biomass can vary according to the source(s) of the biomass. For example, forest-type (woody) residues can require a different manner of storage and transportation than baled corn leaves and stems.

Ensilage refers to a process of storing and preserving biomass to produce silage. Silage can be used as fodder or feed for livestock. Silage can be used for downstream processing; for example, to produce saccharification and/or fermentation end-products.

A biomass to be ensiled can be a forage crop. A forage crop can be a crop that is grown to be utilized by grazing or harvesting as a whole crop. Harvested forage crops can be referred to as forage. In one embodiment, the biomass to be ensiled comprises one or more of grasses, herbaceous legumes, tree legumes, corns or maize, alfalfas, sorghums or sweet sorghums, oats (Avena sativa), rice (e.g., Oryza sativa (Asian rice) or Oryza glaberrima (African rice)), wheat, barley (Hordeum vulgare L.), millets, triticale, ryes (Secale cereale), buckwheat (Fagopyrum esculentum), fonios (e.g., white fonio (Digitaria exilis) or black fonio (Digitaria iburua), quinoa (Chenopodium quinoa), or a combination thereof.

In one embodiment, a biomass to be ensiled comprises one or more grasses; for example, the biomass can comprise one or more of common bentgrass (Agrostis capillaris), creeping bentgrass (Agrostis stolonifera), Andropogon hallii, Brachiaria decumbens, Brachiara humidicola, Bothriochloa pertusa, Bothrioochloa bladhii, Heteropogon contortus, Themeda triandra, Guinea grass (Panicum maximum), Cenchrus ciliaris, Melinis minutiflora, Setaria sphacelata, Chloris guyana, Cynodon dactylon, Paspalum dilatatum, Hyparrhenia rufa, Echinochloa pyrmaidalis, Leersia hexandra, Hymenachne amplexicaulis, Entolasia imbricate, intermediate wheatgrass (Thinopyrum intermedium), Kentucky bluegrass or smooth meadow-grass (Poa pratensis), rough meadow-grass (Poa trivialis), Texas bluegrass (Poa arachnifera), Bromegrass or brome, false oat-grass (Arrhenatherum elatius), red fescue (Festuca rubra), Meadow fescue (Festuca pratensis), Tall fescue (Festuca arundinacea), orchard grass or cock's-foot (Dactylis glomerate), Reed canary-grass (Phalaris arundinacea), annual or Italian ryegrass (Lolium multiflorum), perennial ryegrass (Lolium perenne), or timothy-grass or timothy (Phleum pratense).

In another embodiment, a biomass to be ensiled comprises one or more herbaceous legume; for example, the biomass can comprise one or more of Stylosanthes scabra, Stylosanthes humilis, Chamaecrista rotundifolia, Macroptilium atropurpeum, Macroptilium bracteatum, Medicago truncatula, Glycine wightii, Clitoria ternatea, Arachis pintoi, Vigna parkeri, alfalfa or lucerne (Medicago sativa), bird's-foot trefoil (Lotus corniculatus), alsike clover (Trifolium hybridum), crimson clover (Trifolium incarnatum), red clover (Trifolium pratense), white clover (Trifolium repens), sainfoin (Onobrychis viciifolia), sweetclover or melilot, common vetch or tare (Vicia sativa), hairy vetch (Vicia villosa), bitter vetch (Vicia ervilia), Vicia articulate Hornem, or Vicia narbonensis.

In another embodiment, a biomass to be ensiled can comprise one or more tree legumes; for example, the biomass can comprise one or more of Leucaena leucocephala, Albizia lebbeck, or Acacia aneura.

In another embodiment, a biomass to be ensiled can comprise one or more corns or maizes; for example, the biomass can comprise one or more of flour corn (Zea mays var. amylacea), popcorn (Zea mays var. everta), dent corn (Zea mays var. indentata), flint corn (Zea mays var. indurata), sweet corn (Zea mays var. saccharata and Zea mays var. rugosa), waxy corn (Zea mays var. ceratina), amylomaize (Zea mays), pod corn (Zea mays var. tunicata Larrañaga), or striped maize (Zea mays var. japonica). The biomass can comprise cereal grains (e.g., corn kernels) and/or corn stover (e.g., corn stalk, corn leaves, corn husks, corn cobs, corn straw).

In another embodiment, a biomass to be ensiled can comprise one or more alfalfas; for example, the biomass can comprise one or more of Medicago sativa subsp. ambigua, Medicago sativa subsp. microcarpa, Medicago sativa subsp. sativa, or Medicago sativa subsp. varia.

In another embodiment, a biomass to be ensiled can comprise one or more sorghums or sweet sorghums; for example, the biomass can comprise one or more of Sorghum almum, Sorghum amplum, Sorghum angustum, Sorghum arundinaceum, Sorghum bicolor, Sorghum bicolor subsp. drummondii, Sorghum brachypodum, Sorghum bulbosum, Sorghum burmahicum, Sorghum ecarinatum, Sorghum exstans, Sorghum grande, Sorghum halepense, Sorghum interjectum, Sorghum intrans, Sorghum laxiflorum, Sorghum leiocladum, Sorghum macrospermum, Sorghum matarankense, Sorghum nitidum, Sorghum plumosum, Sorghum propinquum, Sorghum purpureosericeum, Sorghum stipoideum, Sorghum timorense, Sorghum trichocladum, Sorghum versicolor, Sorghum verticiliflorum, or Sorghum vulgare var. technicum.

In another embodiment, a biomass to be ensiled can comprise one or more wheat species; for example, the biomass can comprise one or more of Triticum aestivum, Triticum aethiopicum, Triticum araraticum, Triticum boeoticum, Triticum carthlicum, Triticum compactum, Triticum dicoccoides, Triticum dicoccum, Triticum durum, Triticum ispahanicum, Triticum karamyschevii, Triticum macha, Triticum militinae, Triticum monococcum, Triticum polonicum, Triticum spelta, Triticum sphaerococcum, Triticum timopheevii, Triticum turanicum, Triticum turgidum, Triticum urartu, Triticum vavilovii, or Triticum zhukovskyi.

In another embodiment, a biomass to be ensiled can comprise one or more millets; for example, the biomass can comprise one or more of pearl millet (Pennisetum glaucum), foxtail millet (Setaria italica), common millet (Panicum miliaceum), ringer millet (Eleusine coracana), Indian barnyard millet or sawa millet (Echinochloa frumentacea), Japanese barnyard millet (Echinochloa esculenta), Kodo millet (Paspalum scrobiculatum), little millet (Panicum sumatrense), Guinea millet (Brachiaria deflexa), or browntop millet (Urochloa ramosa).

Ensilage

Ensilage refers to a process of storing and preserving biomass to produce silage. Silage can be used as fodder or feed for livestock. Silage can be used for downstream processing; for example, to produce saccharification and/or fermentation end-products. Three goals of ensiling biomass can include reducing oxygen content, acidification of the biomass, and control of water content.

The process of ensiling, storing and feeding-out biomass can be described as occurring in 6 phases.

Phase I

During the initial ensiling process, freshly cut plant material (biomass/feedstock), and naturally occurring aerobic microorganisms (e.g., bacteria, yeast, mould) found on plant material surfaces, can continue to respire within a container (e.g., silo or other storage structure). Plant and microbial respiration can utilize oxygen trapped within and between the biomass particles at the time of ensiling. This phase can be undesirable since plant and aerobic microbial respiration can consume soluble carbohydrates, thus reducing the yield of the soluble carbohydrates. Reduced yields of the soluble carbohydrates can reduce anaerobic fermentation by anaerobic bacteria in proceeding phases, reduce the nutritional value of the silage as animal feed, or reduce the yield of saccharification or fermentation end-products during downstream processing of the silage. Plant and microbial respiration during phase I can reduce the oxygen levels to produce anaerobic conditions. Plant and bacterial respiration during phase I can also produce water, carbon dioxide, and heat in the biomass. Excessive heat build-up (e.g., resulting from an extended Phase I period) can reduce the digestibility of nutrients such as proteins.

Plant proteins can also be broken down during phase I. Proteins can be first reduced to amino acids and then to ammonia and amines. Up to 50 percent of the total plant protein may be broken down during this process. The extent of protein breakdown (proteolysis) is dependent on the rate of pH decline in the silage. Acidifying the silage can reduce the activity of the enzymes that break down proteins.

Phase I can end once the oxygen has been eliminated from the biomass, or reduced below levels to support aerobic respiration. Phase I can last for a period of time of from a few hours to several weeks. One goal of the ensiling process can be to reduce the oxygen levels in the ensiled biomass in order to reduce the duration of phase I. Factors that can affect the oxygen levels include crop maturity, moisture levels, chop length, and container filling speed.

Phase II

Phase II can begin once oxygen levels in the ensiled biomass are reduced enough to produce an aerobic environment. Phase II can include anaerobic fermentation where the growth and development of acetic acid-producing bacteria occurs. These bacteria can ferment soluble carbohydrates and produce acetic acid as an end product. Acetic acid can be utilized by ruminants. Acetic acid can reduce the pH of the ensiled biomass. Reducing the pH of the ensiled biomass can promote further fermentation phases. As the pH of the ensiled mass falls below 5.0, the acetic bacteria can decline in numbers as this pH level can inhibit their growth. Phase II can last for a period of time of about 72 hours or less (e.g., about 24 hours).

Phase III

The increasing acid inhibits acetic bacteria and brings Phase II to an end. The lower pH can enhance the growth and development of another anaerobic group of bacteria, those producing lactic acid.

Phase IV

Phase IV can be a continuation of Phase III. During phase IV, lactic-acid bacteria can grow and increase in number. Lactic acid bacteria can ferment soluble carbohydrates and produce lactic acid. Lactic acid can comprise greater than 60 percent of the total silage organic acids produced. Lactic acid can also be utilized by cattle as an energy source. Phase IV can be the longest phase in the ensiling process. Phase IV can continue until the pH of the ensiled biomass is low enough to inhibit the growth of all bacteria. When this pH is reached, the can be considered to be in a preserved state. Production of lactic acid can be beneficial to the preservation of the ensiled biomass; however, it can be desirable to limit lactic acid production when ensiling biomass for downstream processing to saccharification and/or fermentation processes. Lactic acid can be carried through and/or inhibit a downstream process such as a saccharification and/or fermentation process. For example, lactic acid can inhibit yeast fermentation. Ensiled biomass that contains lactic acid can require a wash step prior to a downstream saccharification and/or fermentation process. The wash step can be both time-consuming and water consuming and can lead to a reduced yields of soluble sugars from the ensiled biomass.

Phase V

The final pH of the ensiled biomass can depend on the type of biomass being ensiled and/or on the conditions at the time of ensiling. For example, haylage can reach a final pH of around 4.5 and the final pH of corn silage can be about 4.0. Forages ensiled at higher moisture levels (e.g., greater than 70 percent) can undergo a different version of Phase IV. Instead of lactic acid producing bacteria developing, clostridia bacteria can grow in the silage. These anaerobic bacteria can produce butyric acid rather than lactic acid, which can result in sour silage. With this type of fermentation, the final pH may be 5.0 or above.

Phase VI

Phase VI can refer to the silage as it is being fed out from the storage structure. During this phase, secondary aerobic decomposition can occur. Secondary aerobic decomposition can cause a loss of silage dry matter. Secondary aerobic decomposition can occur on any surface of the silage that is exposed to oxygen while in storage in the container. High populations of yeast and mold or the mishandling of stressed crops can lead to losses due to aerobic deterioration of the silage.

In one aspect, disclosed herein are methods and compositions that increase the yield of downstream saccharification and/or fermentation processes from ensiled biomass through improvements in the process of ensiling biomass.

A first goal in ensiling biomass can be to reduce or remove oxygen from the ensiled biomass. Removal of oxygen can be beneficial because removal of oxygen can limit or reduce aerobic respiration. The plant cells of harvested biomass can continue to respire provided there is adequate moisture and oxygen. Removal of oxygen can also reduce or eliminate growth of, and therefore, aerobic respiration of, aerobic microorganisms such as yeasts, molds, and aerobic bacteria. During aerobic respiration, plant sugars (e.g., starches) can be oxidized to produce carbon dioxide, water, and heat. Limiting aerobic respiration by removing oxygen can therefore be beneficial by increasing the yield of plant sugars (e.g., starches) from the biomass. Limiting aerobic respiration by removing oxygen can also be beneficial by decreasing heat buildup in the ensiled biomass. Heat buildup in ensiled biomass can reduce the yields of nutrients such as proteins. Temperatures greater than 110° F. can result in the formation of Maillard reaction products (nitrogen-containing compounds that can be formed by reactions between amino acids and reducing sugars), which can reduce the yield of proteins or sugars from the silage. Generally, high density packing with acidic conditions can increase the stability of any silage. Removing oxygen can be accomplished, for example, by reducing the biomass is size and packing the biomass to a sufficiently high bulk density in a container (e.g., an air-tight container). Removing oxygen can be accomplished by displacing oxygen with another gas. Removing oxygen can be accomplished through the action of plant and/or aerobic microbial respiration. Removing oxygen can be accomplished by the addition of oxygen scavenging chemicals or compounds.

A second goal in ensiling biomass can be to acidify the biomass. Reduction of the pH of the ensiled biomass and subsequent maintenance of this condition can ensure long-term stability of the silage biomass. Properly acidified silage can remain relatively stable for indefinite length of time, provided that oxygen cannot gain access to the silage mass. Acidifying biomass can prevent the growth and respiration of microorganism such as bacteria, yeasts, and molds. If oxygen is allowed access to silage, populations of yeasts and molds can increase their respiration processes, potentially causing substantial dry matter losses. Similar processes can occur when the silage is removed for use. Biomass can be acidified through the anaerobic fermentation of plant sugars to produce organic acids such as lactic acid, acetic acid, butyric acid, citric acid, oxalic acid, uric acid, or formic acid. Anaerobic fermentation can be by bacterial species already in the biomass. Anaerobic fermentation can be by microbial species from cultures that are added to the biomass.

Biomass can be acidified through the direct addition of acids, such as strong acids or weak acids. Strong acids that can be added to ensiled biomass include sulfuric acid (H₂SO₄), hydrochloric acid (HCl), nitric acid (HNO₃), hydrobromic acid (HBr), hydroiodic acid (HI), or perchloric acid (HClO₄). Weak acids that can be added to ensiled biomass can include acetic acid (CH₃COOH), oxalic acid (H₂C₂O₄), lactic acid (C₃H₆O₃), formic acid (HCOOH), propionic acid (C₃H₆O₂), butyric acid (C₄H₈O₂), uric acid (C₅H₄N₄O₃), and citric acid (C₆H₈O₇). Ensiled biomass can be acidified by the addition of chemical agents or compounds that can form acids through chemical reactions with other compounds; for example, sulfur dioxide can react with water to produce sulfurous acid (H₂SO₃). Ensiled biomass can be acidified through a combination of direct addition of acids and bacterial production of acids. Ensiled biomass can also be acidified by the direct addition of acids to a pH that prevents or inhibits anaerobic fermentation by bacteria or other microorganisms. Direct acidification of ensiled biomass can reduce the amount of acids required in a downstream pretreatment or hydrolysis process. Direct acidification can increase the amount of soluble sugars in the ensiled biomass, which can increase the rate of anaerobic fermentation, thus increasing the rate at which the ensiled biomass is acidified.

A third goal in ensiling biomass can be to control the moisture level of the ensiled biomass. High moisture levels can cause silage effluent. High moisture levels can promote the growth of microbial species (e.g., Clostridium species). Clostridial fermentation can produce butyric acid and ammonia nitrogen, which can, for example, cause a rancid odor. High moisture levels can make it more difficult to acidify the ensiled biomass. Low moisture levels can make it more difficult to remove the oxygen from the ensiled biomass. Low moisture levels can inhibit microbial fermentation, thereby reducing the production of acids. Low moisture biomass can contain higher levels of molds or yeasts, which can cause heating of the biomass and a loss of biomass dry weight. This can reduce yields in downstream saccharification and/or fermentation reactions. The moisture levels of ensiled biomass can be controlled by wilting or drying cut biomass crops prior to cutting and packing in a container. The moisture levels of ensiled biomass can be controlled by the addition of water and/or acids to the biomass; for example, adding from about 5 to 6 gallons of water per ton of ensiled biomass can increase the moisture content of the biomass by about 1%. The moisture levels of ensiled biomass can be controlled by the addition of dried biomass; for example, coarsely ground, chopped, and dried cereal grains, alfalfa, or grasses can decrease the moisture content of ensiled biomass by about 5% for each 150 to 200 lbs of material added per ton of ensiled biomass. The moisture levels of ensiled biomass can be controlled by harvesting the biomass at the appropriate moisture levels.

In one embodiment, a biomass is reduced in size prior to packing in a container. In one embodiment, the biomass is reduced in size by chopping, cutting, shredding, chipping, grinding, hammer-milling, or a combination thereof. Different types of biomass (e.g., different crops) can require different packing techniques. Some grasses, for example, can be packed densely with little or no treatment following harvesting. Wood chips, however, can present a problem because of air spaces between them. Chopping, hammer-milling, or other techniques can be utilized to ensure proper packing of biomass to help eliminate oxygen. In one embodiment, the biomass is reduced to an average size of from about 0.1 cm to about 2 cm. For example, the biomass can be reduced to an average size of about 0.1-2 cm, 0.1-1.5 cm, 0.1-1.2 cm, 0.1-1 cm, 0.1-0.8 cm, 0.1-0.6 cm, 0.1-0.3 cm, 0.3-1.5 cm, 0.3-1.2 cm, 0.3-1 cm, 0.3-0.8 cm, 0.3-0.6 cm, 0.6-1.2 cm, 0.6-1 cm, 0.6-0.8 cm, 0.8-1.2 cm, 0.8-1 cm, 1-1.2 cm, 0.1 cm, 0.15 cm, 0.2 cm, 0.25 cm, 0.3 cm, 0.35 cm, 0.4 cm, 0.45 cm, 0.5 cm, 0.55 cm, 0.6 cm, 0.65 cm, 0.7 cm, 0.75 cm, 0.8 cm, 0.85 cm, 0.9 cm, 0.95 cm, 1 cm, 1.05 cm, 1.1 cm, 1.15 cm, 1.2 cm, 1.25 cm, 1.3 cm, 1.35 cm, 1.4 cm, 1.45 cm, 1.5 cm, 1.55 cm, 1.6 cm, 1.65 cm, 1.7 cm, 1.75 cm, 1.8 cm, 1.85 cm, 1.9 cm, 1.95 cm, or 2 cm prior to packing in the container. In one embodiment, the biomass is reduced in size to from about 0.6 cm to about 1.2 cm.

In one embodiment, a biomass is packed in a container at a bulk density of from about 10 lbs/ft³ to about 100 lbs/ft³, during an ensiling process. For example, the biomass can be packed at a bulk density of about 10-100 lbs/ft³, 10-80 lbs/ft³, 10-60 lbs/ft³, 10-50 lbs/ft³, 10-40 lbs/ft³, 10-30 lbs/ft³, 10-20 lbs/ft³, 20-100 lbs/ft³, 20-80 lbs/ft³, 20-60 lbs/ft³, 20-50 lbs/ft³, 20-40 lbs/ft³, 20-30 lbs/ft³, 30-100 lbs/ft³, 30-80 lbs/ft³, 30-60 lbs/ft³, 30-50 lbs/ft³, 30-40 lbs/ft³, 40-100 lbs/ft³, 40-80 lbs/ft³, 40-60 lbs/ft³, 40-50 lbs/ft³, 50-100 lbs/ft³, 50-80 lbs/ft³, 50-60 lbs/ft³, 60-100 lbs/ft³, 60-80 lbs/ft³, 80-100 lbs/ft³, 10 lbs/ft³, 11 lbs/ft³, 12 lbs/ft³, 13 lbs/ft³, 14 lbs/ft³, 15 lbs/ft³, 16 lbs/ft³, 17 lbs/ft³, 18 lbs/ft³, 19 lbs/ft³, 20 lbs/ft³, 21 lbs/ft³, 22 lbs/ft³, 23 lbs/ft³, 24 lbs/ft³, 25 lbs/ft³, 26 lbs/ft³, 27 lbs/ft³, 28 lbs/ft³, 29 lbs/ft³, 30 lbs/ft³, 31 lbs/ft³, 32 lbs/ft³, 33 lbs/ft³, 34 lbs/ft³, 35 lbs/ft³, 36 lbs/ft³, 37 lbs/ft³, 38 lbs/ft³, 39 lbs/ft³, 40 lbs/ft³, 41 lbs/ft³, 42 lbs/ft³, 43 lbs/ft³, 44 lbs/ft³, 45 lbs/ft³, 46 lbs/ft³, 47 lbs/ft³, 48 lbs/ft³, 49 lbs/ft³, 50 lbs/ft³, 51 lbs/ft³, 52 lbs/ft³, 53 lbs/ft³, 54 lbs/ft³, 55 lbs/ft³, 56 lbs/ft³, 57 lbs/ft³, 58 lbs/ft³, 59 lbs/ft³, 60 lbs/ft³, 61 lbs/ft³, 62 lbs/ft³, 63 lbs/ft³, 64 lbs/ft³, 65 lbs/ft³, 66 lbs/ft³, 67 lbs/ft³, 68 lbs/ft³, 69 lbs/ft³, 70 lbs/ft³, 71 lbs/ft³, 72 lbs/ft³, 73 lbs/ft³, 74 lbs/ft³, 75 lbs/ft³, 76 lbs/ft³, 77 lbs/ft³, 78 lbs/ft³, 79 lbs/ft³, 80 lbs/ft³, 81 lbs/ft³, 82 lbs/ft³, 83 lbs/ft³, 84 lbs/ft³, 85 lbs/ft³, 86 lbs/ft³, 87 lbs/ft³, 88 lbs/ft³, 89 lbs/ft³, 90 lbs/ft³, 91 lbs/ft³, 92 lbs/ft³, 93 lbs/ft³, 94 lbs/ft³, 95 lbs/ft³, 96 lbs/ft³, 97 lbs/ft³, 98 lbs/ft³, 99 lbs/ft³, or 100 lbs/ft³. The container can be an air-tight container. The container can be a storage silo (e.g., a tower silo, a bunker silo, a bag silo, etc.).

In one embodiment, one or more gasses are used to displace oxygen when ensiling biomass. The one or more gasses can include CO₂ (carbon dioxide), N₂ (nitrogen), Ar (argon), He (helium), deoxygenated air, SO₂ (sulfur dioxide), NO₂ (nitrogen dioxide), or any other gas. In one embodiment, the one or more gasses used to displace oxygen comprise sulfur dioxide. In another embodiment, the one or more gasses used to displace oxygen comprise carbon dioxide.

A biomass can be ensiled at a moisture content of from about 30% to about 80%. For example, the moisture content of ensiled biomass can be about 30-80%, 30-70%, 30-65%, 30-60%, 30-55%, 30-50%, 50-70%, 50-65%, 50-60%, 50-55%, 55-65%, 55-60%, 60-65%, 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 75%, or 80%. In one embodiment, the moisture content of ensiled biomass is from about 50% to about 70%. In another embodiment, the moisture content of ensiled biomass is from about 55% to about 65%. In another embodiment, the moisture content of ensiled biomass is about 65%. In one embodiment, the moisture content of ensiled biomass is adjusted by the addition of water and or acids. In another embodiment, the moisture content of ensiled biomass is adjusted by the addition of one or more additives. In one embodiment, the one or more additives comprise dried biomass. The dried biomass can be cereal grains, alfalfa, grasses, or a combination thereof. The dried biomass can be coarsely ground or chopped. In one embodiment, the biomass is dried or wilted after harvesting but before ensiling.

A biomass can be ensiled at an elevated temperature; for example, to activate thermophilic enzymes and promote autohydrolysis. The elevated temperature can be from about 40° C. to about 110° C.; for example, about 40-60° C., 40-50° C., 50-110° C., 50-100° C., 50-90° C., 50-80° C., 50-70° C., 50-60° C., 60-110° C., 60-100° C., 60-90° C., 60-80° C., 60-70° C., 70-110° C., 70-100° C., 70-90° C., 70-80° C., 80-110° C., 80-100° C., 80-90° C., 90-110° C., 90-100° C., 100-110° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., 100° C., 101° C., 102° C., 103° C., 104° C., 105° C., 106° C., 107° C., 108° C., 109° C., or 110° C.

In one embodiment, an ensiled biomass is acidified by the addition of one or more acids. In one embodiment, the one or more acids comprise strong acids and/or weak acids. In one embodiment, the one or more acids comprise one or more strong acids such as sulfuric acid (H₂SO₄), hydrochloric acid (HCl), nitric acid (HNO₃), hydrobromic acid (HBr), hydroiodic acid (HI), or perchloric acid (HClO₄). In one embodiment, the one or more acids comprise one or more weak acids such as acetic acid (CH₃COOH), oxalic acid (H₂C₂O₄), lactic acid (C₃H₆O₃), formic acid (HCOOH), propionic acid (C₃H₆O₂), butyric acid (C₄H₈O₂), uric acid (C₅H₄N₄O₃), or citric acid (C₆H₈O₇). In one embodiment, sulfuric acid is added to the ensiled biomass. In another embodiment, formic acid is added to the ensiled biomass. In another embodiment, propionic acid is added to the ensiled biomass. In one embodiment, formic acid and propionic acid are added to the ensiled biomass.

An ensiled biomass can be acidified to a pH of from about 3 to about 6; for example, the biomass can be acidified to a pH of about 3-6,3-5.5, 3-5,3-4.5, 3-4,3-3.5, 3.5-6, 3.5-5.5, 3.5-5, 3.5-4.5, 3.5-4,4-6, 4-5.5, 4-5,4-4.5, 4.5-6, 4.5-5.5, 4.5-5,5-6, 5-5.5, 5.5-6,3,3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, or 6. In one embodiment, the biomass is acidified by the action of endogenous microorganisms such as bacteria. In one embodiment, the biomass is acidified by the action of microorganisms (e.g., anaerobic bacteria) that are added to the biomass. In one embodiment, the biomass is acidified by the addition of one or more acids. In one embodiment, the one or more acids comprise sulfuric acid. In another embodiment, the one or more acids comprise formic acid. In another embodiment, the one or more acids comprise propionic acid. In one embodiment, the ensiled biomass is acidified by the addition of sulfuric dioxide gas. In another embodiment, the ensiled biomass is acidified by the addition of formic acid and sulfur dioxide. In one embodiment, the ensiled biomass is acidified by both the addition of one or more acids and the anaerobic fermentation of one or more bacteria.

Sulfur dioxide (SO₂) can be added to biomass during an ensiling process. Sulfur dioxide can affect all phases of the ensiling process. Sulfur dioxide gas can reduce the levels of oxygen in the biomass (e.g., by displacing oxygen in the biomass and/or scavenging oxygen in the biomass), which can reduce the amount of aerobic fermentation. Sulfur dioxide can react with water to form sulfurous acid, which can help to acidify the ensiled biomass and inhibit microbial growth (e.g., growth of gram negative bacteria and/or Clostridia). Dissociation of a hydrogen ion from sulfurous acid forms a bisulfate ion, which can acidify the biomass and/or inhibit the growth of molds and yeast. The use of SO₂ gas in the ensiling process can eliminate an initial washing step and/or replace some of the acid (e.g., SO₄) in dilute acid hydrolysis, thus reducing the cost of downstream pretreatment, saccharification, and/or fermentation processes.

Without being bound by theory, the following equations show potential modes of action of SO₂ in the ensiling process:

1. S+O₂═SO₂ [Sulfur is an oxygen scavenger—helps prevent mold and yeast growth]

2. SO₂+H₂O (moisture in forage)=H₂SO₃ (Sulfurous acid) [Sulfurous acid can inhibit gram negative bacteria.]

3. H₂SO₃←→H⁺(ions-acid)+HSO₃ (Bisulfite ion) [Bisulfites can inhibit mold and yeast, which can reduce competition with the good crop bacteria for available plant sugars and/or increase the yield of downstream saccharification and/or fermentation processes.]

Sulfur dioxide gas can be added to a biomass during an ensiling process. Sulfur dioxide can be added to the biomass in an amount of from about 0.1% by weight to about 20% by dry matter weight; for example, about 0.1-20%, 0.1-10%, 0.1-5%, 0.1-3%, 0.1-2%, 0.1-1%, 0.1-0.5%, 0.5-10%, 0.5-5%, 0.5-3%, 0.5-2%, 0.5-1%, 1-5%, 1-3%, 1-2%, 2-5%, 2-3%, 3-5%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.5%, 6%, 6.5%, 7%, 7.5%, 8%, 8.5%, 9%, 9.5%, 10%, 11%, 12%, 13%, 14%, 15%, 16%, 17%, 18%, 19%, or 20%. In one embodiment, the amount of sulfur dioxide added to the biomass is from about 1% to about 5%. In another embodiment, the amount of sulfur dioxide added to the biomass is from about 2% to about 3%.

Formic acid can be added to a biomass during an ensiling process. Formic acid can acidify the biomass and can reduce or inhibit aerobic and/or anaerobic fermentation processes. Formic acid can form breakdown products such as carbon monoxide and/or carbon dioxide, which can displace oxygen in the biomass. Formic acid can reduce the amount of butyric acid formed during anaerobic fermentation of the biomass during the ensiling process. Formic acid can reduce the amount of lactic acid formed during the ensiling process. Formic acid can increase the amount and/or rate of lactic acid production during the ensiling process. The effects of formic acid on butyric and/or lactic acid production can be concentration dependent. Formic acid can be added to the biomass in an amount of from about 0.1% to about 20% by dry matter weight; for example, about 0.1-20%, 0.1-10%, 0.1-5%, 0.1-3%, 0.1-2%, 0.1-1%, 0.1-0.5%, 0.5-10%, 0.5-5%, 0.5-3%, 0.5-2%, 0.5-1%, 1-5%, 1-3%, 1-2%, 2-5%, 2-3%, 3-5%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.5%, 6%, 6.5%, 7%, 7.5%, 8%, 8.5%, 9%, 9.5%, 10%, 11%, 12%, 13%, 14%, 15%, 16%, 17%, 18%, 19%, or 20%. In one embodiment, the amount of formic acid added to the biomass is from about 1% to about 5%. In another embodiment, the amount of formic acid added to the biomass is from about 2% to about 3%.

Propionic acid can be added to a biomass during an ensiling process. Propionic acid can acidify the biomass and can reduce or inhibit aerobic and/or anaerobic fermentation processes. Propionic acid can inhibit the growth of microorganisms such as mold and/or bacteria. Propionic acid can reduce the amount of butyric acid formed during anaerobic fermentation of the biomass during the ensiling process. Propionic acid can reduce the amount of lactic acid formed during the ensiling process. Propionic acid can increase the amount and/or rate of lactic acid production during the ensiling process. The effects of propionic acid on butyric and/or lactic acid production can be concentration dependent. Propionic acid can be added to the biomass in an amount of from about 0.1% by weight to about 20% by wet matter weight; for example, about 0.1-20%, 0.1-10%, 0.1-5%, 0.1-3%, 0.1-2%, 0.1-1%, 0.1-0.5%, 0.5-10%, 0.5-5%, 0.5-3%, 0.5-2%, 0.5-1%, 1-5%, 1-3%, 1-2%, 2-5%, 2-3%, 3-5%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.5%, 6%, 6.5%, 7%, 7.5%, 8%, 8.5%, 9%, 9.5%, 10%, 11%, 12%, 13%, 14%, 15%, 16%, 17%, 18%, 19%, or 20%. In one embodiment, the amount of propionic acid added to the biomass is from about 0.1% to about 5%. In another embodiment, the amount of propionic acid added to the biomass is from about 0.5% to about 1%.

In one embodiment, formic acid and/or sulfur dioxide are added to a biomass during an ensiling process. The addition of formic acid and SO₂ can reduce oxygen levels in the ensiled mass. The sulfur in SO₂, in the presence of the moisture and plant acids from the biomass can produce sulfur compounds (gases) and formic acid can breakdown to CO₂, which can slow the growth of bacteria and molds. In one embodiment, formic acid in an amount from about 0.1% to about 20% by dry matter weight and sulfur dioxide is added in an amount of from about 0.1% to about 20% by dry matter weight. In another embodiment, formic acid in an amount from about 1% to about 5% by dry matter weight and sulfur dioxide is added in an amount of from about 1% to about 5% by dry matter weight.

### Other Storage Methods

Ensilement can be used for long-term storage. However, the methods described herein can also be used for short-term storage of biomass to prevent loss of fermentable carbohydrates. The breakdown of the biomass prior to pretreatment can be dependent on the amount of oxygen present, the moisture levels, and the acidity of the biomass. Storage can be done in any type of closed container or in a controlled environment. CO₂, for example, can be used to replace the atmosphere, slowing initial fermentation until the biomass pretreatment is initiated. CO₂ does not have to be washed out and will dissipate during pretreatment. Additionally, CO₂ will help reduce the pH of the biomass. Other weak acids, such as formic acid, can be added, or SO₂ as described herein, to further increase acidity. The moisture levels can be controlled through harvesting at proper times, drying, addition additional dried material, or adding moisture as needed. This can help decrease levels of undesirable microorganisms without requiring further treatment prior to hydrolyzation.

Pretreatment of Ensiled Biomass

The initial steps of pretreatment of an ensiled or stored biomass can involve washing of the biomass. The washing can be to remove residual lactic acid produced by lactic acid fermentation, which can interfere with pretreatment and fermentation processes, especially yeast fermentation. This can be both a time-consuming and water consuming step. The use of other weak acid treatments can be an advantage in that this step can be eliminated. As described supra, propionic acid, formic acid and/or sulfur dioxide can be used. The use of propionic acid, formic acid and/or sulfur dioxide during ensiling can enable silage to be pretreated without a washing step; for example, by reducing the amount of lactic acid produced during the ensiling process. The use of propionic acid, formic acid and/or sulfur dioxide during ensiling can increase the yield of saccharification and/or fermentation products from downstream processing (e.g., pretreating, hydrolyzing, fermenting) of silage; for example, by reducing the loss of plant sugars to aerobic respiration of plant cells and/or yeasts and molds and/or by reducing the loss of plant sugars to anaerobic respiration by bacteria. Propionic acid, formic acid and/or sulfur dioxide can escape during pretreatment, thus speed up the pretreatment process considerably while saving water.

In one embodiment, an ensiled biomass is produced through reduction of oxygen and treatment with propionic and/or formic acid and/or SO₂ gas and stored indefinitely. CO₂ can also be used as described supra to replace the atmosphere surrounding the biomass. The biomass can then be pretreated without a washing step to remove lactic acid. The biomass can comprises starch, cellulose, hemicellulose, lignocellulose, or a combination thereof that can be hydrolyzed to produce monomeric sugars during a pretreatment/hydrolysis process. The biomass can be pretreated according to any of the methods disclosed herein; for example, by dilute acid, hot water treatment, stream explosion, or an alkaline pretreatment. The biomass can be pretreated using a combination of techniques; for example, the biomass can be pretreated using hot water or stream explosion followed by alkaline treatment.

In one embodiment, an increased yield of monomeric sugars is produced from the pretreatment and/or hydrolysis of silage produced by ensiling with propionic acid and/or formic acid and/or sulfur dioxide in comparison to a yield from silage produced without using propionic acid and/or formic acid and/or sulfur dioxide. The increased yield can be from about 1% to about 50% higher; for example, the increased yield can be about 1-50%, 1-25%, 1-15%, 1-10%, 1-5%, 5-50%, 5-25%, 5-15%, 5-10%, 10-50%, 10-25%, 10-15%, 15-50%, 15-25%, 25-50%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% higher.

### Pretreatment of Feedstock

Cellulose is a linear polymer of glucose where the glucose units are connected via β(1->4) linkages. Hemicellulose is a branched polymer of a number of sugar monomers including glucose, xylose, mannose, galactose, rhamnose and arabinose, and can have sugar acids such as mannuronic acid and galacturonic acid present as well. Lignin is a cross-linked, racemic macromolecule of mostlyp-coumaryl alcohol, conferyl alcohol and sinapyl alcohol. These three polymers occur together in lignocellusic materials in plant biomass. The different characteristics of the three polymers can make hydrolysis of the combination difficult as each polymer tends to shield the others from enzymatic attack.

In one embodiment, methods are provided for the pretreatment of feedstock used in the fermentation and production of the biofuels and chemicals. The pretreatment steps can include mechanical, thermal, pressure, chemical, thermochemical, and/or biochemical tests pretreatment prior to being used in a bioprocess for the production of fuels and chemicals, but untreated biomass material can be used in the process as well. Mechanical processes can reduce the particle size of the biomass material so that it can be more conveniently handled in the bioprocess and can increase the surface area of the feedstock to facilitate contact with chemicals/biochemicals/biocatalysts.

Mechanical processes can also separate one type of biomass material from another. The biomass material can also be subjected to thermal and/or chemical pretreatments to render plant polymers more accessible. Multiple steps of treatment can also be used.

Mechanical processes include, are not limited to, washing, soaking, milling, size reduction, screening, shearing, size classification and density classification processes. Chemical processes include, but are not limited to, bleaching, oxidation, reduction, acid treatment, base treatment, sulfite treatment, acid sulfite treatment, basic sulfite treatment, ammonia treatment, and hydrolysis. Thermal processes include, but are not limited to, sterilization, ammonia fiber expansion or explosion (“AFEX”), steam explosion, holding at elevated temperatures, pressurized or unpressurized, in the presence or absence of water, and freezing. Biochemical processes include, but are not limited to, treatment with enzymes, including enzymes produced by genetically-modified plants, and treatment with microorganisms. Various enzymes that can be utilized include cellulase, amylase, β-glucosidase, xylanase, gluconase, and other polysaccharases; lysozyme; laccase, and other lignin-modifying enzymes; lipoxygenase, peroxidase, and other oxidative enzymes; proteases; and lipases. One or more of the mechanical, chemical, thermal, thermochemical, and biochemical processes can be combined or used separately. Such combined processes can also include those used in the production of paper, cellulose products, microcrystalline cellulose, and cellulosics and can include pulping, kraft pulping, acidic sulfite processing. The feedstock can be a side stream or waste stream from a facility that utilizes one or more of these processes on a biomass material, such as cellulosic, hemicellulosic or lignocellulosic material. Examples include paper plants, cellulosics plants, distillation plants, cotton processing plants, and microcrystalline cellulose plants. The feedstock can also include cellulose-containing or cellulosic containing waste materials. The feedstock can also be biomass materials, such as wood, grasses, corn, starch, or sugar, produced or harvested as an intended feedstock for production of ethanol or other products such as by biocatalysts.

In another embodiment, a method can utilize a pretreatment process disclosed in U.S. Patents and Patent Applications US20040152881, US20040171136, US20040168960, US20080121359, US20060069244, US20060188980, US20080176301, 5693296, 6262313, US20060024801, 5969189, 6043392, US20020038058, US5865898, US5865898, US6478965, 5986133, or US20080280338, each of which is incorporated by reference herein in its entirety.

In another embodiment, the AFEX process is be used for pretreatment of biomass. In a preferred embodiment, the AFEX process is used in the preparation of cellulosic, hemicellulosic or lignocellulosic materials for fermentation to ethanol or other products. The process generally includes combining the feedstock with ammonia, heating under pressure, and suddenly releasing the pressure. Water can be present in various amounts. The AFEX process has been the subject of numerous patents and publications.

In another embodiment, the pretreatment of biomass comprises the addition of calcium hydroxide to a biomass to render the biomass susceptible to degradation. Pretreatment comprises the addition of calcium hydroxide and water to the biomass to form a mixture, and maintaining the mixture at a relatively high temperature. Alternatively, an oxidizing agent, selected from the group consisting of oxygen and oxygen-containing gasses, can be added under pressure to the mixture. Examples of carbon hydroxide treatments are disclosed in U.S. Pat. No. 5,865,898 to Holtzapple and S. Kim and M. T. Holzapple, Bioresource Technology, 96, (2005) 1994, incorporated by reference herein in its entirety.

In one embodiment, pretreatment of biomass comprises dilute acid hydrolysis. Example of dilute acid hydrolysis treatment are disclosed in T. A. Lloyd and C. E Wyman, Bioresource Technology, (2005) 96, 1967), incorporated by reference herein in its entirety.

In another embodiment, pretreatment of biomass comprises pH controlled liquid hot water treatment. Examples of pH controlled liquid hot water treatments are disclosed in N. Mosier et al., Bioresource Technology, (2005) 96, 1986, incorporated by reference herein in its entirety.

In one embodiment, pretreatment of biomass comprises aqueous ammonia recycle process (ARP). Examples of aqueous ammonia recycle process are described in T. H. Kim and Y. Y. Lee, Bioresource Technology, (2005)96, 2007, incorporated by reference herein in its entirety.

In one embodiment, the above-mentioned methods have two steps: a pretreatment step that leads to a wash stream, and an enzymatic hydrolysis step of pretreated-biomass that produces a hydrolysate stream. In the above methods, the pretreatment step can include acid hydrolysis, hot water pretreatment, steam explosion or alkaline reagent based methods (AFEX, ARP, and lime pretreatments). Dilute acid and hot water treatment methods can be used to solubilize all or a portion of the hemicellulose. Methods employing alkaline reagents can be used remove all, most, or a portion of the lignin during the pretreatment step. As a result, the wash stream from the pretreatment step in the former methods contains mostly hemicellulose-based sugars, whereas this stream has mostly lignin for the high-pH methods. The subsequent enzymatic hydrolysis of the residual biomass leads to mixed sugars (C5 and C6) in the alkali based pretreatment methods, while glucose is the major product in the hydrolyzate from the low and neutral pH methods. In one embodiment, the treated material is additionally treated with catalase or another similar chemical, chelating agents, surfactants, and other compounds to remove impurities or toxic chemicals or further release polysaccharides.

In one embodiment, pretreatment of biomass comprises ionic liquid (IL) pretreatment. Biomass can be pretreated by incubation with an ionic liquid, followed by IL extraction with a wash solvent such as alcohol or water. The treated biomass can then be separated from the ionic liquid/wash-solvent solution by centrifugation or filtration, and sent to the saccharification reactor or vessel. Examples of ionic liquid pretreatment are disclosed in US publication No. 2008/0227162, incorporated herein by reference in its entirety.

In another embodiment, a method can utilize a pretreatment process disclosed in U.S. Pat. No. 4,600,590 to Dale, U.S. Pat. No. 4,644,060 to Chou, U.S. Pat. No. 5,037,663 to Dale. U.S. Pat. No. 5,171,592 to Holtzapple, et al., et al., U.S. Pat. No. 5,939,544 to Karstens, et al., U.S. Pat. No. 5,473,061 to Bredereck, et al., U.S. Pat. No. 6,416,621 to Karstens., U.S. Pat. No. 6,106,888 to Dale, et al., U.S. Pat. No. 6,176,176 to Dale, et al., PCT publication WO2008/020901 to Dale, et al., Felix, A., et al., Anim. Prod. 51, 47-61 (1990)., Wais, A. C., Jr., et al., Journal of Animal Science, 35, No. 1, 109-112 (1972), which are incorporated herein by reference in their entireties.

Alteration of the pH of a pretreated feedstock can be accomplished by washing the feedstock (e.g., with water) one or more times to remove an alkaline or acidic substance, or other substance used or produced during pretreatment. Washing can comprise exposing the pretreated feedstock to an equal volume of water 2, 3, 4, 5, 6, 7, 8, 9, 10, 11, 12, 13, 14, 15, 16, 17, 18, 19, 20, 21, 22, 23, 24, 25 or more times. In another embodiment, a pH modifier can be added. For example, an acid, a buffer, or a material that reacts with other materials present can be added to modulate the pH of the feedstock. In one embodiment, more than one pH modifier can be used, such as one or more bases, one or more bases with one or more buffers, one or more acids, one or more acids with one or more buffers, or one or more buffers. When more than one pH modifiers are utilized, they can be added at the same time or at different times. Other non-limiting exemplary methods for neutralizing feedstocks treated with alkaline substances have been described, for example in U.S. Pat. Nos. 4,048,341; 4,182,780; and 5,693,296.

In one embodiment, one or more acids can be combined, resulting in a buffer. Suitable acids and buffers that can be used as pH modifiers include any liquid or gaseous acid that is compatible with the microorganism. Non-limiting examples include peroxyacetic acid, sulfuric acid, lactic acid, citric acid, phosphoric acid, and hydrochloric acid. In some instances, the pH can be lowered to neutral pH or acidic pH, for example a pH of 7.0, 6.5, 6.0, 5.5, 5.0, 4.5, 4.0, or lower. In some embodiments, the pH is lowered and/or maintained within a range of about pH 4.5 to about 7.1, or about 4.5 to about 6.9, or about pH 5.0 to about 6.3, or about pH 5.5 to about 6.3, or about pH 6.0 to about 6.5, or about pH 5.5 to about 6.9 or about pH 6.2 to about 6.7.

In another embodiment, biomass can be pre-treated at an elevated temperature and/or pressure. In one embodiment, biomass is pre treated at a temperature range of 20° C. to 400° C. In another embodiment, biomass is pretreated at a temperature of about 20° C., 25° C., 30° C., 35° C., 40° C., 45° C., 50° C., 55° C., 60° C., 65° C., 80° C., 90° C., 100° C., 120° C., 150° C., 200° C., 250° C., 300° C., 350° C., 400° C. or higher. In another embodiment, elevated temperatures are provided by the use of steam, hot water, or hot gases. In one embodiment, steam can be injected into a biomass containing vessel. In another embodiment, the steam, hot water, or hot gas can be injected into a vessel jacket such that it heats, but does not directly contact the biomass.

In another embodiment, a biomass can be treated at an elevated pressure. In one embodiment, biomass is pre treated at a pressure range of about 1 psi to about 30 psi. In another embodiment, biomass is pre treated at a pressure or about 1 psi, 2 psi, 3 psi, 4 psi, 5 psi, 6 psi, 7 psi, 8 psi, 9 psi, 10 psi, 12 psi, 15 psi, 18 psi, 20 psi, 22 psi, 24 psi, 26 psi, 28 psi, 30 psi or more. In some embodiments, biomass can be treated with elevated pressures by the injection of steam into a biomass containing vessel. In one embodiment, the biomass can be treated to vacuum conditions prior or subsequent to alkaline or acid treatment or any other treatment methods provided herein.

In one embodiment, alkaline or acid pretreated biomass is washed (e.g. with water (hot or cold) or other solvent such as alcohol (e.g. ethanol)), pH neutralized with an acid, base, or buffering agent (e.g. phosphate, citrate, borate, or carbonate salt) or dried prior to fermentation. In one embodiment, the drying step can be performed under vacuum to increase the rate of evaporation of water or other solvents. Alternatively, or additionally, the drying step can be performed at elevated temperatures such as about 20° C., 25° C., 30° C., 35° C., 40° C., 45° C., 50° C., 55° C., 60° C., 65° C., 80° C., 90° C., 100° C., 120° C., 150° C., 200° C., 250° C., 300° C. or more.

In one embodiment, the pretreatment step includes a step of solids recovery. The solids recovery step can be during or after pretreatment (e.g., acid or alkali pretreatment), or before the drying step. In one embodiment, the solids recovery step can include the use of a sieve, filter, screen, or a membrane for separating the liquid and solids fractions. In one embodiment, a suitable sieve pore diameter size ranges from about 0.001 microns to 8 mm, such as about 0.005 microns to 3 mm or about 0.01 microns to 1 mm. In one embodiment, a sieve pore size has a pore diameter of about 0.01 microns, 0.02 microns, 0.05 microns, 0.1 microns, 0.5 microns, 1 micron, 2 microns, 4 microns, 5 microns, 10 microns, 20 microns, 25 microns, 50 microns, 75 microns, 100 microns, 125 microns, 150 microns, 200 microns, 250 microns, 300 microns, 400 microns, 500 microns, 750 microns, 1 mm or more. In one embodiment, biomass (e.g. corn stover) is processed or pretreated prior to fermentation. In one embodiment, a method of pre-treatment includes but is not limited to, biomass particle size reduction, such as for example shredding, milling, chipping, crushing, grinding, or pulverizing. In one embodiment, biomass particle size reduction can include size separation methods such as sieving, or other suitable methods known in the art to separate materials based on size. In one embodiment, size separation can provide for enhanced yields. In one embodiment, separation of finely shredded biomass (e.g. particles smaller than about 8 mm in diameter, such as, 8, 7.9, 7.7, 7.5, 7.3, 7, 6.9, 6.7, 6.5, 6.3, 6, 5.9, 5.7, 5.5, 5.3, 5, 4.9, 4.7, 4.5, 4.3, 4, 3.9, 3.7, 3.5, 3.3, 3, 2.9, 2.7, 2.5, 2.3, 2, 1.9, 1.7, 1.5, 1.3, 1, 0.9, 0.8, 0.7, 0.6, 0.5, 0.4, 0.3, 0.2, or 0.1 mm) from larger particles allows the recycling of the larger particles back into the size reduction process, thereby increasing the final yield of processed biomass. In one embodiment, a fermentative mixture is provided which comprises a pretreated lignocellulosic feedstock comprising less than about 50% of a lignin component present in the feedstock prior to pretreatment and comprising more than about 60% of a hemicellulose component present in the feedstock prior to pretreatment; and a microorganism capable of fermenting a five-carbon sugar, such as xylose, arabinose or a combination thereof, and a six-carbon sugar, such as glucose, galactose, mannose or a combination thereof. In some instances, pretreatment of the lignocellulosic feedstock comprises adding an alkaline substance which raises the pH to an alkaline level, for example NaOH. In one embodiment, NaOH is added at a concentration of about 0.5% to about 2% by weight of the feedstock. In one embodiment, pretreatment also comprises addition of a chelating agent.

Hydrolysis

In one embodiment, the biomass hydrolyzing unit provides useful advantages for the conversion of biomass to biofuels and chemical products. One advantage of this unit is its ability to produce monomeric sugars from multiple types of biomass, including mixtures of different biomass materials, and is capable of hydrolyzing polysaccharides and higher molecular weight saccharides to lower molecular weight saccharides. In one embodiment, the hydrolyzing unit utilizes a pretreatment process and a hydrolytic enzyme which facilitates the production of a sugar stream containing a concentration of a monomeric sugar or several monomeric sugars derived from cellulosic and/or hemicellulosic polymers. Examples of biomass material that can be pretreated and hydrolyzed to manufacture sugar monomers include, but are not limited to, cellulosic, hemicellulosic, lignocellulosic materials; pectins; starches; wood; paper; agricultural products; forest waste; tree waste; tree bark; leaves; grasses; sawgrass; woody plant matter; non-woody plant matter; carbohydrates; starch; inulin; fructans; glucans; corn; sugar cane; sorghum, other grasses; bamboo, algae, and material derived from these materials. This ability to use a very wide range of pretreatment methods and hydrolytic enzymes gives distinct advantages in biomass fermentations. Various pretreatment conditions and enzyme hydrolysis can enhance the extraction of sugars from biomass, resulting in higher yields, higher productivity, greater product selectivity, and/or greater conversion efficiency.

In one embodiment, the enzyme treatment is used to hydrolyze various higher saccharides (higher molecular weight) present in biomass to lower saccharides (lower molecular weight), such as in preparation for fermentation by biocatalysts such as yeasts to produce ethanol, hydrogen, or other chemicals such as organic acids including succinic acid, formic acid, acetic acid, and lactic acid. These enzymes and/or the hydrolysate can be used in fermentations to produce various products including fuels, and other chemicals.

In one example, the process for converting biomass material into ethanol includes pretreating the biomass material (e.g., “feedstock”), hydrolyzing the pretreated biomass to convert polysaccharides to oligosaccharides, further hydrolyzing the oligosaccharides to monosaccharides, and converting the monosaccharides to biofuels and chemical products. Enzymes such as cellulases, polysaccharases, lipases, proteases, ligninases, and hemicellulases, help produce the monosaccharides can be used in the biosynthesis of fermentation end-products. Biomass material that can be utilized includes woody plant matter, non-woody plant matter, cellulosic material, lignocellulosic material, hemicellulosic material, carbohydrates, pectin, starch, inulin, fructans, glucans, corn, algae, sugarcane, other grasses, switchgrass, bagasse, wheat straw, barley straw, rice straw, corncobs, bamboo, citrus peels, sorghum, high biomass sorghum, seed hulls, and material derived from these. The final product can then be separated and/or purified, as indicated by the properties for the desired final product. In some instances, compounds related to sugars such as sugar alcohols or sugar acids can be utilized as well.

Chemicals that can be used in the methods disclosed herein can be purchased from a commercial supplier, such as Sigma-Aldrich. Additionally, commercial enzyme cocktails (e.g. Accellerase™ 1000, CelluSeb-TL, CelluSeb-TS, Cellic™, CTec, STARGEN™, Maxalig™ Spezyme®, Distillase®, G-Zyme®, Fermenzyme®, Fermgen™, GC 212, or Optimash™) or any other commercial enzyme cocktail can be purchased from vendors such as Specialty Enzymes & Biochemicals Co., Genencor, or Novozymes. Alternatively, enzyme cocktails can be prepared by growing one or more organisms such as for example a fungi (e.g. a Trichoderma, a Saccharomyces, a Pichia, a White Rot Fungus etc.), a bacteria (e.g. a Clostridium, or a coliform bacterium, a Zymomonas bacterium, Sacharophagus degradans etc.) in a suitable medium and harvesting enzymes produced therefrom. In some embodiments, the harvesting can include one or more steps of purification of enzymes.

In one embodiment, treatment of biomass comprises enzyme hydrolysis. In one embodiment, a biomass is treated with an enzyme or a mixture of enzymes, e.g., endoglucanases, exoglucanases, cellobiohydrolases, cellulase, beta-glucosidases, glycoside hydrolases, glycosyltransferases, lyases, esterases, amylases, glucoamylases, and proteins containing carbohydrate-binding modules. In one embodiment, the enzyme or mixture of enzymes is one or more individual enzymes with distinct activities. In another embodiment, the enzyme or mixture of enzymes can be enzyme domains with a particular catalytic activity. For example, an enzyme with multiple activities can have multiple enzyme domains, including for example glycoside hydrolases, glycosyltransferases, lyases and/or esterases catalytic domains.

In one embodiment, enzymes that degrade polysaccharides are used for the hydrolysis of biomass and can include enzymes that degrade cellulose, namely, cellulases. Examples of some cellulases include endocellulases and exo-cellulases that hydrolyze beta-1,4-glucosidic bonds.

In one embodiment, enzymes that degrade polysaccharides are used for the hydrolysis of biomass and can include enzymes that have the ability to degrade hemicellulose, namely, hemicellulases. Hemicellulose can be a major component of plant biomass and can contain a mixture of pentoses and hexoses, for example, D-xylopyranose, L-arabinofuranose, D-mannopyranose, Dglucopyranose, D-galactopyranose, D-glucopyranosyluronic acid and other sugars. In one embodiment, enzymes that degrade polysaccharides are used for the hydrolysis of biomass and can include enzymes that have the ability to degrade pectin, namely, pectinases. In plant cell walls, the cross-linked cellulose network can be embedded in a matrix of pectins that can be covalently cross-linked to xyloglucans and certain structural proteins. Pectin can comprise homogalacturonan (HG) or rhamnogalacturonan (RH).

In one embodiment, hydrolysis of biomass includes enzymes that can hydrolyze starch. Enzymes that hydrolyze starch include alpha-amylase, glucoamylase, beta-amylase, exo-alpha-1,4-glucanase, and pullulanase.

In one embodiment, hydrolysis of biomass comprises hydrolases that can include enzymes that hydrolyze chitin. In another embodiment, hydrolases can include enzymes that hydrolyze lichen, namely, lichenase.

In one embodiment, after pretreatment and/or hydrolysis by any of the above methods the feedstock contains cellulose, hemicellulose, soluble oligomers, simple sugars, lignin, volatiles and ash. The parameters of the hydrolysis can be changed to vary the concentration of the components of the pretreated feedstock. For example, a hydrolysis can be chosen so that the concentration of soluble C5 saccharides is high and the concentration of lignin is low after hydrolysis. Examples of parameters of the hydrolysis include temperature, pressure, time, concentration, composition and pH.

In one embodiment, the parameters of the pretreatment and hydrolysis are changed to vary the concentration of the components of the pretreated feedstock such that concentration of the components in the pretreated and hydrolyzed feedstock is optimal for fermentation with a microbe such as a yeast or bacterium microbe.

In one embodiment, the parameters of the pretreatment are changed to encourage the release of the components of a genetically modified feedstock such as enzymes stored within a vacuole to increase or complement the enzymes synthesized by biocatalyst to produce optimal release of the fermentable components during hydrolysis and fermentation.

In one embodiment, the parameters of the pretreatment and hydrolysis are changed such that concentration of accessible cellulose in the pretreated feedstock is 1%, 5%, 10%, 12%, 13%, 14%, 15%, 16%, 17%, 19%, 20%, 30%, 40% or 50%. In one embodiment, the parameters of the pretreatment are changed such that concentration of accessible cellulose in the pretreated feedstock is 5% to 30%. In one embodiment, the parameters of the pretreatment are changed such that concentration of accessible cellulose in the pretreated feedstock is 10% to 20%.

In one embodiment, the parameters of the pretreatment are changed such that concentration of hemicellulose in the pretreated feedstock is 1%, 5%, 10%, 12%, 13%, 14%, 15%, 16%, 17%, 19%, 20%, 21%, 22%, 23%, 24%, 25%, 26%, 27%, 28%, 29%, 30%, 40% or 50%. In one embodiment, the parameters of the pretreatment are changed such that concentration of hemicellulose in the pretreated feedstock is 5% to 40%. In one embodiment, the parameters of the pretreatment are changed such that concentration of hemicellulose in the pretreated feedstock is 10% to 30%.

In one embodiment, the parameters of the pretreatment and hydrolysis are changed such that concentration of soluble oligomers in the pretreated feedstock is 1%, 10%, 15%, 20%, 25%, 30%, 35%, 40%, 45%, 50%, 55%, 60%, 65%, 70%, 75%, 80%, 85%, 90%, 95%, or 99%. Examples of soluble oligomers include, but are not limited to, cellobiose and xylobiose. In one embodiment, the parameters of the pretreatment are changed such that concentration of soluble oligomers in the pretreated feedstock is 30% to 90%. In one embodiment, the parameters of the pretreatment and/or hydrolysis are changed such that concentration of soluble oligomers in the pretreated feedstock is 45% to 80%.

In one embodiment, the parameters of the pretreatment and hydrolysis are changed such that concentration of simple sugars in the pretreated feedstock is 1%, 5%, 10%, 12%, 13%, 14%, 15%, 16%, 17%, 19%, 20%, 30%, 40% or 50%. In one embodiment, the parameters of the pretreatment and hydrolysis are changed such that concentration of simple sugars in the pretreated feedstock is 0% to 20%. In one embodiment, the parameters of the pretreatment and hydrolysis are changed such that concentration of simple sugars in the pretreated feedstock is 0% to 5%. Examples of simple sugars include, but are not limited to, C5 and C6 monomers and dimers.

In one embodiment, the parameters of the pretreatment are changed such that concentration of lignin in the pretreated and/or hydrolyzed feedstock is 1%, 5%, 10%, 12%, 13%, 14%, 15%, 16%, 17%, 19%, 20%, 30%, 40% or 50%. In one embodiment, the parameters of the pretreatment and/or hydrolysis are changed such that concentration of lignin in the pretreated feedstock is 0% to 20%. In one embodiment, the parameters of the pretreatment and/or hydrolysis are changed such that concentration of lignin in the pretreated feedstock is 0% to 5%. In one embodiment, the parameters of the pretreatment and hydrolysis are changed such that concentration of lignin in the pretreated and/or hydrolyzed feedstock is less than 1% to 2%. In one embodiment, the parameters of the pretreatment and/or hydrolysis are changed such that the concentration of phenolics is minimized.

In one embodiment, the parameters of the pretreatment and/or hydrolysis are changed such that concentration of furfural and low molecular weight lignin in the pretreated and/or hydrolyzed feedstock is less than 10%, 9%, 8%, 7%, 6%, 5%, 4%, 3%, 2%, or 1%. In one embodiment, the parameters of the pretreatment and/or hydrolysis are changed such that concentration of furfural and low molecular weight lignin in the pretreated and/or hydrolyzed feedstock is less than 1% to 2%.

In one embodiment, the parameters of the pretreatment and/or hydrolysis are changed such that the concentration of simple sugars is at least 75% to 85%, and the concentration of lignin is 0% to 5% and the concentration of furfural and low molecular weight lignin in the pretreated feedstock is less than 1% to 2%.

In one embodiment, the parameters of the pretreatment and/or hydrolysis are changed to obtain a high concentration of hemicellulose and a low concentration of lignin. In one embodiment, the parameters of the pretreatment and/or hydrolysis are changed to obtain a high concentration of hemicellulose and a low concentration of lignin such that concentration of the components in the pretreated stock is optimal for fermentation with a microbe such as biocatalyst.

In one embodiment, more than one of these steps can occur at any given time. For example, hydrolysis of the pretreated feedstock and hydrolysis of the oligosaccharides can occur simultaneously, and one or more of these can occur simultaneously to the conversion of monosaccharides to a fuel or chemical.

In another embodiment, an enzyme can directly convert the polysaccharide to monosaccharides. In some instances, an enzyme can hydrolyze the polysaccharide to oligosaccharides and the enzyme or another enzyme can hydrolyze the oligosaccharides to monosaccharides.

In another embodiment, the enzymes can be added to the fermentation or they can be produced by microorganisms present in the fermentation. In one embodiment, the microorganism present in the fermentation produces some enzymes. In another embodiment, enzymes are produced separately and added to the fermentation.

For the overall conversion of pretreated biomass to final product to occur at high rates, the enzymes for each conversion step can be present with sufficiently high activity. If one of these enzymes is missing or is present in insufficient quantities, the production rate of an end product can be reduced. The production rate can also be reduced if the microorganisms responsible for the conversion of monosaccharides to product only slowly take up monosaccharides and/or have only limited capability for translocation of the monosaccharides and intermediates produced during the conversion to end product. Additions of fractions obtained from pretreatment and/or pretreatment and hydrolysis can increase initial or overall growth rates. In another embodiment, oligomers are taken up slowly by a biocatalyst, necessitating an almost complete conversion of polysaccharides and oligomers to monomeric sugars.

In another embodiment, the enzymes of the method are produced by a biocatalyst, including a range of hydrolytic enzymes suitable for the biomass materials used in the fermentation methods. In one embodiment, a biocatalyst is grown under conditions appropriate to induce and/or promote production of the enzymes needed for the saccharification of the polysaccharide present. The production of these enzymes can occur in a separate vessel, such as a seed fermentation vessel or other fermentation vessel, or in the production fermentation vessel where ethanol production occurs. When the enzymes are produced in a separate vessel, they can, for example, be transferred to the production fermentation vessel along with the cells, or as a relatively cell free solution liquid containing the intercellular medium with the enzymes. When the enzymes are produced in a separate vessel, they can also be dried and/or purified prior to adding them to the hydrolysis or the production fermentation vessel. The conditions appropriate for production of the enzymes are frequently managed by growing the cells in a medium that includes the biomass that the cells will be expected to hydrolyze in subsequent fermentation steps. Additional medium components, such as salt supplements, growth factors, and cofactors including, but not limited to phytate, amino acids, and peptides can also assist in the production of the enzymes utilized by the microorganism in the production of the desired products.

Fermentation

Enhanced rates of fermentation can be achieved using single or blended feedstocks comprising a mixture of non-cellulosic polysaccharides (e.g., starch) and one or more monosaccharides (e.g., in a C6 enriched hydrolyzate) in comparison to fermentation of the non-cellulosic polysaccharides without the one or more monosaccharides. The enhanced rates of fermentation can be from about 1% higher to about 100% higher; for example, about 1-100%, 1-75%, 1-50%, 1-25%, 1-10%, 10-100%, 10-75%, 10-50%, 10-25%, 25-100%, 25-75%, 25-50%, 50-100%, 50-75%, 75-100%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, 95%, or 100% higher.

Increased yields of one or more fermentation end-products can be achieved using blended feedstocks comprising a mixture of non-cellulosic polysaccharides (e.g., starch) and one or more monosaccharides (e.g., from a C6 enriched hydrolyzate) in comparison to fermentation of the non-cellulosic polysaccharides without the one or more monosaccharides. The increased yields of one or more fermentation end-products can be from about 1% higher to about 100% higher; for example, about 1-100%, 1-75%, 1-50%, 1-25%, 1-10%, 10-100%, 10-75%, 10-50%, 10-25%, 25-100%, 25-75%, 25-50%, 50-100%, 50-75%, 75-100%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, 95%, or 100% higher.

A blended feedstock can be prepared by combining a first biomass with one or more monosaccharides. The first biomass can comprise non-cellulosic polysaccharides such as starch. The one or more monosaccharides can be produced by the pretreatment and/or hydrolysis of a second biomass. The second biomass can comprise cellulose, hemicellulose, or lignocellulose. The pretreatment and/or hydrolysis of the second biomass can produce a C6 enriched hydrolyzate. In one embodiment, the one or more monosaccharides (e.g., from a C6 enriched hydrolyzate) are at a concentration that differs from the concentration of sugars in the first biomass by less than about +/−50%, 40%, 30%, 20%, 15%, 10%, 5%, or 1%, wherein the concentration of sugars in the first biomass assumes complete hydrolysis of the first biomass to monomers.

A blended feedstock can be prepared by combining a first biomass with one or more monosaccharides. The first biomass can comprise non-cellulosic polysaccharides such as starch. The one or more monosaccharides can be produced by the pretreatment and/or hydrolysis of a second biomass. The second biomass can comprise cellulose, hemicellulose, or lignocellulose. The pretreatment and/or hydrolysis of the second biomass can produce a C6 enriched hydrolyzate. In one embodiment, the first biomass and the one or more monosaccharides (e.g., from a C6 enriched hydrolyzate) are combined in a 50:50, 55:45, 60:40:, 65:35, 70:30, 75:35, 80:20, 85:15, 90:10, 95:5, or 99:1 ratio.

Exposing microorganisms such as bacteria or yeast to hypertonic solution can cause an efflux of cellular water into the medium. In order to counteract the outflow of water molecules during growth, microorganisms can produce and accumulate one or more osmoregulatory molecules such as polyhydroxy compounds. (e.g., see Nevoit and Stahl (1997) FEMS Microbiology Review 21:231-241 and Parekh and Pandey (1985) Biotechnology and Bioengineering 27: 1089-1091, each of which is incorporated by reference in its entirety). During ethanolic fermentation of starch-containing compounds, microorganisms such as yeast can redirect part of the carbon released during enzymatic hydrolysis of starch to polyols or sugar alcohols (e.g., glycerol) instead of ethanol. This can occur, for example, when glucose is overly abundant during the conversion of starch to glucose monomers. Environmental factors affecting these pathways can include oxygen availability, type of nitrogen source, osmotic pressure, heat and pH. For example, when glucose is overly abundant, a high osmotic pressure can shift metabolism to the production of glycerol. Therefore, it may be possible to maintain high ethanol production with controlled and slowed feeding of starch to reduce glycerol production.

In one embodiment, the concentration of monosaccharides at the start of a fermentation or simultaneous saccharification and fermentation reaction can be less than about 100 g/L; for example, less than about 100 g/L, 90 g/L, 80 g/L, 70 g/L, 60 g/L, 50 g/L, 40 g/L, 30 g/L, 25 g/L, 20 g/L, 15 g/L, 10 g/L, 9 g/L, 8 g/L, 7 g/L, 6 g/L, 5 g/L, 4 g/L, 3 g/L, 2 g/L, or 1 g/L. In another embodiment, the concentration of monosaccharides at the start of a fermentation or simultaneous saccharification and fermentation reaction can be from about 1 g/L to about 100 g/L; for example, about 1-100 g/L, 1-75 g/L, 1-50 g/L, 1-25 g/L, 1-10 g/L, 10-100 g/L, 10-75 g/L, 10-50 g/L, 10-25 g/L, 25-100 g/L, 25-75 g/L, 25-50 g/L, 50-100 g/L, 50-75 g/L, or 75-100 g/L.

The present disclosure also provides a fermentative mixture comprising: a cellulosic feedstock pre-treated with an alkaline or acid substance and at a temperature of from about 80° C. to about 120° C.; subsequently hydrolyzed with an enzyme mixture, and a microorganism capable of fermenting a five-carbon sugar and/or a six-carbon sugar. In one embodiment, the five-carbon sugar is xylose, arabinose, or a combination thereof. In one embodiment, the six-carbon sugar is glucose, galactose, mannose, or a combination thereof. In one embodiment, the alkaline substance is NaOH. In some embodiments, NaOH is added at a concentration of about 0.5% to about 2% by weight of the feedstock. In one embodiment, the acid is equal to or less than 2% HCl or H₂SO₄. In one embodiment, the microorganism is a Rhodococcus strain, a Clostridium strain, a Trichoderma strain, a Saccharomyces strain, a Zymomonas strain, or another microorganism suitable for fermentation of biomass. In another embodiment, the fermentation process comprises fermentation of the biomass using a microorganism that is Clostridium phytofermentans, Clostridium algidixylanolyticum, Clostridium xylanolyticum, Clostridium cellulovorans, Clostridium cellulolyticum, Clostridium thermocellum, Clostridium josui, Clostridium papyrosolvens, Clostridium cellobioparum, Clostridium hungatei, Clostridium cellulosi, Clostridium stercorarium, Clostridium termitidis, Clostridium thermocopriae, Clostridium celerecrescens, Clostridium polysaccharolyticum, Clostridium populeti, Clostridium lentocellum, Clostridium chartatabidum, Clostridium aldrichii, Clostridium herbivorans, Acetivibrio cellulolyticus, Bacteroides cellulosolvens, Caldicellulosiruptor saccharolyticum, Rhodococcus opacus, Ruminococcus albus, Ruminococcus flavefaciens, Fibrobacter succinogenes, Eubacterium cellulosolvens, Butyrivibrio fibrisolvens, Anaerocellum thermophilum, Halocella cellulolytica, Thermoanaerobacterium thermosaccharolyticum, Sacharophagus degradans, or Thermoanaerobacterium saccharolyticum. In still another embodiment, the microorganism is genetically modified to enhance activity of one or more hydrolytic enzymes, such as a genetically-modified Saccaromyces cerevisae.

In one embodiment, a wild type or a genetically-improved microorganism can be used for chemical production by fermentation. Methods to produce a genetically-improved strain can include genetic modification, mutagenesis, and adaptive processes, such as directed evolution. For example, yeasts can be genetically-modified to ferment C5 sugars. Other useful yeasts are species of Candida, Cryptococcus, Debaryomyces, Deddera, Hanseniaspora, Kluyveromyces, Pichia, Schizosaccharomyces, and Zygosaccharomyces. Rhodococus strains, such as Rhodococcus opacus variants are a source of triacylglycerols and other storage lipids. (See, e.g., Walternann, et al., Microbiology 146:1143-1149 (2000)). Other useful organisms for fermentation include, but are not limited to, yeasts, especially Saccaromyces strains and bacteria such as Clostridium phytofermentans, Thermoanaerobacter ethanolicus, Clostridium thermocellum, Clostridium beijerinickii, Clostridium acetobutylicum, Clostridium tyrobutyricum, Clostridium thermobutyricum, Thermoanaerobacterium saccharolyticum, Thermoanaerobacter thermohydrosulfuricus, Clostridium acetobutylicum, Moorella ssp., Carboxydocella ssp., Zymomonas mobilis, recombinant E. Coli, Klebsiella oxytoca, Rhodococcus opacus and Clostridium beijerickii.

An advantage of yeasts are their ability to grow under conditions that include elevated ethanol concentration, high sugar concentration, low sugar concentration, and/or operate under anaerobic conditions. These characteristics, in various combinations, can be used to achieve operation with long or short fermentation cycles and can be used in combination with batch fermentations, fed batch fermentations, self-seeding/partial harvest fermentations, and recycle of cells from the final fermentation as inoculum.

Examples of yeasts that can be used as a biocatalyst or fermentive microorganism in the methods disclosed herein include but are not limited to, species found in the genus Ascoidea, Brettanomyces, Candida, Cephaloascus, Coccidiascus, Dipodascus, Eremothecium, Galactomyces, Kluyveromyces, Pichia, Saccharomyces, Schizosaccharomyces, Sporopachydermia, Torulaspora, Yarrowia, or Zygosaccharomyces; for example, Ascoidea rebescens, Brettanomyces anomalus, Brettanomyces bruxellensis, Brettanomyces claussenii, Brettanomyces custersianus, Brettanomyces lambicus, Brettanomyces naardenensis, Brettanomyces nanus, Candida albicans, Candida ascalaphidarum, Candida amphixiae, Candida antarctica, Candida argentea, Candida atlantica, Candida atmosphaerica, Candida blattae, Candida carpophila, Candida cerambycidarum, Candida chauliodes, Candida corydali, Candida dosseyi, Candida dubliniensis, Candida ergatensis, Candida fructus, Candida glabrata, Candida fermentati, Candida guilliermondii, Candida haemulonii, Candida insectamens, Candida insectorum, Candida intermedia, Candida jeffresii, Candida kefyr, Candida krusei, Candida lusitaniae, Candida lyxosophila, Candida maltosa, Candida marina, Candida membranifaciens, Candida milleri, Candida oleophila, Candida oregonensis, Candida parapsilosis, Candida quercitrusa, Candida rugosa, Candida sake, Candida shehatea, Candida temnochilae, Candida tenuis, Candida tropicalis, Candida tsuchiyae, Candida sinolaborantium, Candida sojae, Candida subhashii, Candida viswanathii, Candida utilis, Cephaloascus fragrans, Coccidiascus legeri, Dypodascus albidus, Eremothecium cymbalariae, Galactomyces candidum, Galactomyces geotrichum, Kluyveromyces aestuarii, Kluyveromyces africanus, Kluyveromyces bacillisporus, Kluyveromyces blattae, Kluyveromyces dobzhanskii, Kluyveromyces hubeiensis, Kluyveromyces lactis, Kluyveromyces lodderae, Kluyveromyces marxianus, Kluyveromyces nonfermentans, Kluyveromyces piceae, Kluyveromyces sinensis, Kluyveromyces thermotolerans, Kluyveromyces waltii, Kluyveromyces wickerhamii, Kluyveromyces yarrowii, Pichia anomola, Pichia heedii, Pichia guilliermondii, Pichia kluyveri, Pichia membranifaciens, Pichia norvegensis, Pichia ohmeri, Pichia pastoris, Pichia subpelliculosa, Saccharomyces bayanus, Saccharomyces boulardii, Saccharomyces bulderi, Saccharomyces cariocanus, Saccharomyces cariocus, Saccharomyces cerevisiae, Saccharomyces chevalieri, Saccharomyces dairenensis, Saccharomyces ellipsoideus, Saccharomyces eubayanus, Saccharomyces exiguus, Saccharomyces florentinus, Saccharomyces kluyveri, Saccharomyces martiniae, Saccharomyces monacensis, Saccharomyces norbensis, Saccharomyces paradoxus, Saccharomyces pastorianus, Saccharomyces spencerorum, Saccharomyces turicensis, Saccharomyces unisporus, Saccharomyces uvarum, Saccharomyces zonatus, Schizosaccharomyces cryophilus, Schizosaccharomyces japonicus, Schizosaccharomyces octosporus, Schizosaccharomyces pombe, Sporopachydermia cereana, Sporopachydermia lactativora, Sporopachydermia quercuum, Torulaspora delbrueckii, Torulaspora franciscae, Torulaspora globosa, Torulaspora pretoriensis, Yarrowia lipolytica, Zygosaccharomyces bailii, Zygosaccharomyces bisporus, Zygosaccharomyces cidri, Zygosaccharomyces fermentati, Zygosaccharomyces florentinus, Zygosaccharomyces kombuchaensis, Zygosaccharomyces lentus, Zygosaccharomyces mellis, Zygosaccharomyces microellipsoides, Zygosaccharomyces mrakii, Zygosaccharomyces pseudorouxii, or Zygosaccharomyces rouxii, or a variant or genetically modified version thereof.

Examples of bacteria that can be used as a biocatalyst or fermentive microorganism in the methods disclosed herein include but are not limited to any bacterium found in the genus of Butyrivibrio, Ruminococcus, Eubacterium, Bacteroides, Acetivibrio, Caldibacillus, Acidothermus, Cellulomonas, Curtobacterium, Micromonospora, Actinoplanes, Streptomyces, Thermobifida, Thermomonospora, Microbispora, Fibrobacter, Sporocytophaga, Cytophaga, Flavobacterium, Achromobacter, Xanthomonas, Cellvibrio, Pseudomonas, Myxobacter, Escherichia, Klebsiella, Thermoanaerobacterium, Thermoanaerobacter, Geobacillus, Saccharococcus, Paenibacillus, Bacillus, Caldicellulosiruptor, Anaerocellum, Anoxybacillus, Zymomonas, Clostridium; for example, Butyrivibrio fibrisolvens, Ruminococcus flavefaciens, Ruminococcus succinogenes, Ruminococcus albus, Eubacterium cellulolyticum, Bacteroides cellulosolvens, Acetivibrio cellulolyticus, Acetivibrio cellulosolvens, Caldibacillus cellulovorans, Bacillus circulans, Acidothermus cellulolyticus, Cellulomonas cartae, Cellulomonas cellasea, Cellulomonas cellulans, Cellulomonas fimi, Cellulomonas flavigena, Cellulomonas gelida, Cellulomonas iranensis, Cellulomonas persica, Cellulomonas uda, Curtobacterium falcumfaciens, Micromonospora melonosporea, Actinoplanes aurantiaca, Streptomyces reticuli, Streptomyces alboguseolus, Streptomyces aureofaciens, Streptomyces cellulolyticus, Streptomyces flavogriseus, Streptomyces lividans, Streptomyces nitrosporeus, Streptomyces olivochromogenes, Streptomyces rochei, Streptomyces thermovulgaris, Streptomyces viridosporus, Thermobifida alba, Thermobifida fusca, Thermobifida cellulolytica, Thermomonospora curvata, Microbispora bispora, Fibrobacter succinogenes, Sporocytophaga myxococcoides, Cytophaga sp., Flavobacterium johnsoniae, Achromobacter piechaudii, Xanthomonas sp., Cellvibrio vulgaris, Cellvibrio fulvus, Cellvibrio gilvus, Cellvibrio mixtus, Pseudomonas fluorescens, Pseudomonas mendocina, Myxobacter sp. AL-1, Escherichia albertii, Escherichia blattae, Escherichia coli, Escherichia fergusonii, Escherichia hermannii, Escherichia vulneris, Klebsiella granulomatis, Klebsiella oxytoca, Klebsiella pneumonia, Klebsiella terrigena, Thermoanaerobacterium thermosulfurigenes, Thermoanaerobacterium aotearoense, Thermoanaerobacterium polysaccharolyticum, Thermoanaerobacterium zeae, Thermoanaerobacterium xylanolyticum, Thermoanaerobacterium saccharolyticum, Thermoanaerobium brockii, Thermoanaerobacterium thermosaccharolyticum, Thermoanaerobacter thermohydrosulfuricus, Thermoanaerobacter ethanolicus, Thermoanaerobacter brocki, Geobacillus thermoglucosidasius, Geobacillus stearothermophilus, Saccharococcus caldoxylosilyticus, Saccharoccus thermophilus, Paenibacillus campinasensis, Bacillus flavothermus, Anoxybacillus kamchatkensis, Anoxybacillus gonensis, Caldicellulosiruptor acetigenus, Caldicellulosiruptor saccharolyticus, Caldicellulosiruptor kristjanssonii, Caldicellulosiruptor owensensis, Caldicellulosiruptor lactoaceticus, Anaerocellum thermophilum, Clostridium thermocellum, Clostridium cellulolyticum, Clostridium straminosolvens, Clostridium acetobutylicum, Clostridium aerotolerans, Clostridium beijerinckii, Clostridium bifermentans, Clostridium botulinum, Clostridium butyricum, Clostridium cadaveric, Clostridium chauvoei, Clostridium clostridioforme, Clostridium colicanis, Clostridium difficile, Clostridium fallax, Clostridium formicaceticum, Clostridium histolyticum, Clostridium innocuum, Clostridium ljungdahlii, Clostridium laramie, Clostridium lavalense, Clostridium novyi, Clostridium oedematiens, Clostridium paraputrificum, Clostridium perfringens, Clostridium phytofermentans, Clostridium piliforme, Clostridium ramosum, Clostridium scatologenes, Clostridium septicum, Clostridium sordellii, Clostridium sporogenes, Clostridium tertium, Clostridium tetani, Clostridium tyrobutyricum, Clostridium thermobutyricum, Zymomonas mobilis, or a variant or genetically modified version thereof.

In one embodiment, fed-batch fermentation is performed on the treated biomass to produce a fermentation end-product, such as alcohol, ethanol, organic acid, succinic acid, TAG, or hydrogen. In one embodiment, the fermentation process comprises simultaneous hydrolysis and fermentation (SSF) of the biomass using one or more microorganisms such as a Rhodococcus strain, a Clostridium strain, a Trichoderma strain, a Saccharomyces strain, a Zymomonas strain, or another microorganism suitable for fermentation of biomass. In another embodiment, the fermentation process comprises simultaneous hydrolysis and fermentation of the biomass using a microorganism that is Clostridium algidixylanolyticum, Clostridium xylanolyticum, Clostridium cellulovorans, Clostridium cellulolyticum, Clostridium thermocellum, Clostridium josui, Clostridium papyrosolvens, Clostridium cellobioparum, Clostridium hungatei, Clostridium cellulosi, Clostridium stercorarium, Clostridium termitidis, Clostridium thermocopriae, Clostridium celerecrescens, Clostridium polysaccharolyticum, Clostridium populeti, Clostridium lentocellum, Clostridium chartatabidum, Clostridium aldrichii, Clostridium herbivorans, Clostridium phytofermentans, Acetivibrio cellulolyticus, Bacteroides cellulosolvens, Caldicellulosiruptor saccharolyticum, Ruminococcus albus, Ruminococcus flavefaciens, Fibrobacter succinogenes, Eubacterium cellulosolvens, Butyrivibrio fibrisolvens, Anaerocellum thermophilum, Halocella cellulolytica, Thermoanaerobacterium thermosaccharolyticum, Sacharophagus degradans, or Thermoanaerobacterium saccharolyticum.

In one embodiment, the fermentation process can include separate hydrolysis and fermentation (SHF) of a biomass with one or more enzymes, such as a xylanases, endo-1,4-beta-xylanases, xylosidases, beta-D-xylosidases, cellulases, hemicellulases, carbohydrates, glucanases, endoglucanases, endo-1,4-beta-glucanases, exoglucanases, glucosidases, beta-D-glucosidases, amylases, cellobiohydrolases, exocellobiohydrolases, phytases, proteases, peroxidase, pectate lyases, galacturonases, or laccases. In one embodiment, one or more enzymes used to treat a biomass is thermostable. In another embodiment, a biomass is treated with one or more enzymes, such as those provided herein, prior to fermentation. In another embodiment, a biomass is treated with one or more enzymes, such as those provided herein, during fermentation. In another embodiment, a biomass is treated with one or more enzymes, such as those provided herein, prior to fermentation and during fermentation. In another embodiment, an enzyme used for hydrolysis of a biomass is the same as those added during fermentation. In another embodiment, an enzyme used for hydrolysis of biomass is different from those added during fermentation.

In some embodiments, fermentation can be performed in an apparatus such as bioreactor, a fermentation vessel, a stirred tank reactor, or a fluidized bed reactor. In one embodiment, the treated biomass can be supplemented with suitable chemicals to facilitate robust growth of the one or more fermenting organisms. In one embodiment, a useful supplement includes but is not limited to, a source of nitrogen and/or amino acids such as yeast extract, cysteine, or ammonium salts (e.g. nitrate, sulfate, phosphate etc.); a source of simple carbohydrates such as corn steep liquor, and malt syrup; a source of vitamins such as yeast extract; buffering agents such as salts (including but not limited to citrate salts, phosphate salts, or carbonate salts); or mineral nutrients such as salts of magnesium, calcium, or iron. In some embodiments redox modifiers are added to the fermentation mixture including but not limited to cysteine or mercaptoethanol.

In one embodiment, the titer and/or productivity of fermentation end-product production by a microorganism is improved by culturing the microorganism in a medium comprising one or more compounds comprising hexose and/or pentose sugars. In one embodiment, a process comprises conversion of a starting material (such as a biomass) to a biofuel, such as one or more alcohols. In one embodiment, methods can comprise contacting substrate comprising both hexose (e.g. glucose, cellobiose) and pentose (e.g. xylose, arabinose) saccharides with a microorganism that can hydrolyze C5 and C6 saccharides to produce ethanol. In another embodiment, methods can comprise contacting substrate comprising both hexose (e.g. glucose, cellobiose) and pentose (e.g. xylose, arabinose) saccharides with R. opacus to produce TAG.

In some embodiments, batch fermentation with a microorganism of a mixture of hexose and pentose saccharides using the methods disclosed herein can provide uptake rates of about 0.1-8 g/L/h or more of hexose and about 0.1-8 g/L/h or more of pentose (xylose, arabinose, etc.). In some embodiments, batch fermentation with a microorganism of a mixture of hexose and pentose saccharides using the methods disclosed herein can provide uptake rates of about 0.1, 0.2, 0.4, 0.5, 0.6 0.7, 0.8, 1, 2, 3, 4, 5, or 6 g/L/h or more of hexose and about 0.1, 0.2, 0.4, 0.5, 0.6 0.7, 0.8, 1, 2, 3, 4, 5, or 6 g/L/h or more of pentose.

In one embodiment, a method for production of ethanol or another alcohol produces about 10 g/l to 120 gain 40 hours or less. In another embodiment, a method for production of ethanol produces about 10 g/l, 11 g/L, 12 g/L, 13 g/L, 14 g/L, 15 g/L, 16 g/L, 17 g/L, 18 g/L, 19 g/L, 20 g/L, 21 g/L, 22 g/L, 23 g/L, 24 g/L, 25 g/L, 26 g/L, 27 g/L, 28 g/L, 29 g/L, 30 g/L, 31 g/L, 32 g/L, 33 g/L, 34 g/L, 35 g/L, 36 g/L, 37 g/L, 38 g/L, 39 g/L, 40 g/L, 41 g/L, 42 g/L, 43 g/L, 44 g/L, 45 g/L, 46 g/L, 47 g/L, 48 g/L, 49 g/L, 50 g/L, 51 g/L, 52 g/L, 53 g/L, 54 g/L, 55 g/L, 56 g/L, 57 g/L, 58 g/L, 59 g/L, 60 g/L, 61 g/L, 62 g/L, 63 g/L, 64 g/L, 65 g/L, 66 g/L, 67 g/L, 68 g/L, 69 g/L, 70 g/L, 71 g/L, 72 g/L, 73 g/L, 74 g/L, 75 g/L, 76 g/L, 77 g/L, 78 g/L, 79 g/L, 80 g/L, 81 g/L, 82 g/L, 83 g/L, 84 g/L, 85 g/L, 86 g/L, 87 g/L, 88 g/L, 89 g/L, 90 g/L, 91 g/L, 92 g/L, 93 g/L, 94 g/L, 95 g/L, 96 g/L, 97 g/L, 98 g/L, 99 g/L, 100 g/L, 110 g/l, 120 g/l, or more alcohol in 40 hours by the fermentation of biomass. In another embodiment, alcohol is produced by a method comprising simultaneous fermentation of hexose and pentose saccharides. In another embodiment, alcohol is produced by a microorganism comprising simultaneous fermentation of hexose and pentose saccharides.

In another embodiment, the level of a medium component is maintained at a desired level by adding additional medium component as the component is consumed or taken up by the organism. Examples of medium components included, but are not limited to, carbon substrate, nitrogen substrate, vitamins, minerals, growth factors, cofactors, and biocatalysts. The medium component can be added continuously or at regular or irregular intervals. In one embodiment, additional medium component is added prior to the complete depletion of the medium component in the medium. In one embodiment, complete depletion can effectively be used, for example to initiate different metabolic pathways, to simplify downstream operations, or for other reasons as well. In one embodiment, the medium component level is allowed to vary by about 10% around a midpoint, in one embodiment, it is allowed to vary by about 30% around a midpoint, and in one embodiment, it is allowed to vary by 60% or more around a midpoint. In one embodiment, the medium component level is maintained by allowing the medium component to be depleted to an appropriate level, followed by increasing the medium component level to another appropriate level. In one embodiment, a medium component, such as vitamin, is added at two different time points during fermentation process. For example, one-half of a total amount of vitamin is added at the beginning of fermentation and the other half is added at midpoint of fermentation.

In another embodiment, the nitrogen level is maintained at a desired level by adding additional nitrogen-containing material as nitrogen is consumed or taken up by the organism. The nitrogen-containing material can be added continuously or at regular or irregular intervals. Useful nitrogen levels include levels of about 5 to about 10 g/L. In one embodiment, levels of about 1 to about 12 g/L can also be usefully employed. In another embodiment, levels, such as about 0.5, 0.1 g/L or even lower, and higher levels, such as about 20, 30 g/L or even higher are used. In another embodiment, a useful nitrogen level is about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22 23, 24, 25, 26, 27, 28, 29 or 30 g/L. Nitrogen can be supplied as a simple nitrogen-containing material, such as an ammonium compounds (e.g. ammonium sulfate, ammonium hydroxide, ammonia, ammonium nitrate, or any other compound or mixture containing an ammonium moiety), nitrate or nitrite compounds (e.g. potassium, sodium, ammonium, calcium, or other compound or mixture containing a nitrate or nitrite moiety), or as a more complex nitrogen-containing material, such as amino acids, proteins, hydrolyzed protein, hydrolyzed yeast, yeast extract, dried brewer's yeast, yeast hydrolysates, distillers' grains, soy protein, hydrolyzed soy protein, fermentation products, and processed or corn steep powder or unprocessed protein-rich vegetable or animal matter, including those derived from bean, seeds, soy, legumes, nuts, milk, pig, cattle, mammal, fish, as well as other parts of plants and other types of animals. Nitrogen-containing materials useful in various embodiments also include materials that contain a nitrogen-containing material, including, but not limited to mixtures of a simple or more complex nitrogen-containing material mixed with a carbon source, another nitrogen-containing material, or other nutrients or non-nutrients, and AFEX treated plant matter.

In another embodiment, the carbon level is maintained at a desired level by adding sugar compounds or material containing sugar compounds (“Sugar-Containing Material”) as sugar is consumed or taken up by the organism. The sugar-containing material can be added continuously or at regular or irregular intervals. In one embodiment, additional sugar-containing material is added prior to the complete depletion of the sugar compounds available in the medium. In one embodiment, complete depletion can effectively be used, for example to initiate different metabolic pathways, to simplify downstream operations, or for other reasons as well. In one embodiment, the carbon level (as measured by the grams of sugar present in the sugar-containing material per liter of broth) is allowed to vary by about 10% around a midpoint, in one embodiment, it is allowed to vary by about 30% around a midpoint, and in one embodiment, it is allowed to vary by 60% or more around a midpoint. In one embodiment, the carbon level is maintained by allowing the carbon to be depleted to an appropriate level, followed by increasing the carbon level to another appropriate level. In some embodiments, the carbon level can be maintained at a level of about 5 to about 120 g/L. However, levels of about 30 to about 100 g/L can also be usefully employed as well as levels of about 60 to about 80 g/L. In one embodiment, the carbon level is maintained at greater than 25 g/L for a portion of the culturing. In another embodiment, the carbon level is maintained at about 5 g/L, 6 g/L, 7 g/L, 8 g/L, 9 g/L, 10 g/L, 11 g/L, 12 g/L, 13 g/L, 14 g/L, 15 g/L, 16 g/L, 17 g/L, 18 g/L, 19 g/L, 20 g/L, 21 g/L, 22 g/L, 23 g/L, 24 g/L, 25 g/L, 26 g/L, 27 g/L, 28 g/L, 29 g/L, 30 g/L, 31 g/L, 32 g/L, 33 g/L, 34 g/L, 35 g/L, 36 g/L, 37 g/L, 38 g/L, 39 g/L, 40 g/L, 41 g/L, 42 g/L, 43 g/L, 44 g/L, 45 g/L, 46 g/L, 47 g/L, 48 g/L, 49 g/L, 50 g/L, 51 g/L, 52 g/L, 53 g/L, 54 g/L, 55 g/L, 56 g/L, 57 g/L, 58 g/L, 59 g/L, 60 g/L, 61 g/L, 62 g/L, 63 g/L, 64 g/L, 65 g/L, 66 g/L, 67 g/L, 68 g/L, 69 g/L, 70 g/L, 71 g/L, 72 g/L, 73 g/L, 74 g/L, 75 g/L, 76 g/L, 77 g/L, 78 g/L, 79 g/L, 80 g/L, 81 g/L, 82 g/L, 83 g/L, 84 g/L, 85 g/L, 86 g/L, 87 g/L, 88 g/L, 89 g/L, 90 g/L, 91 g/L, 92 g/L, 93 g/L, 94 g/L, 95 g/L, 96 g/L, 97 g/L, 98 g/L, 99 g/L, 100 g/L, 101 g/L, 102 g/L, 103 g/L, 104 g/L, 105 g/L, 106 g/L, 107 g/L, 108 g/L, 109 g/L, 110 g/L, 111 g/L, 112 g/L, 113 g/L, 114 g/L, 115 g/L, 116 g/L, 117 g/L, 118 g/L, 119 g/L, 120 g/L, 121 g/L, 122 g/L, 123 g/L, 124 g/L, 125 g/L, 126 g/L, 127 g/L, 128 g/L, 129 g/L, 130 g/L, 131 g/L, 132 g/L, 133 g/L, 134 g/L, 135 g/L, 136 g/L, 137 g/L, 138 g/L, 139 g/L, 140 g/L, 141 g/L, 142 g/L, 143 g/L, 144 g/L, 145 g/L, 146 g/L, 147 g/L, 148 g/L, 149 g/L, or 150 g/L.

The carbon substrate, like the nitrogen substrate, can be used for cell production and enzyme production, but unlike the nitrogen substrate, the carbon substrate can serve as the raw material for production of fermentation end-products. Frequently, more carbon substrate can lead to greater production of fermentation end-products. In another embodiment, it can be advantageous to operate with the carbon level and nitrogen level related to each other for at least a portion of the fermentation time. In one embodiment, the ratio of carbon to nitrogen is maintained within a range of about 30:1 to about 10:1. In another embodiment, the ratio of carbon nitrogen is maintained from about 20:1 to about 10:1 or more preferably from about 15:1 to about 10:1. In another embodiment, the ratio of carbon nitrogen is about 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, or 1:1.

Maintaining the ratio of carbon and nitrogen ratio within particular ranges can result in benefits to the operation such as the rate of metabolism of carbon substrate, which depends on the amount of carbon substrate and the amount and activity of enzymes present, being balanced to the rate of end product production. Balancing the carbon to nitrogen ratio can, for example, facilitate the sustained production of these enzymes such as to replace those which have lost activity.

In another embodiment, the amount and/or timing of carbon, nitrogen, or other medium component addition can be related to measurements taken during the fermentation. For example, the amount of monosaccharides present, the amount of insoluble polysaccharide present, the polysaccharase activity, the amount of product present, the amount of cellular material (for example, packed cell volume, dry cell weight, etc.) and/or the amount of nitrogen (for example, nitrate, nitrite, ammonia, urea, proteins, amino acids, etc.) present can be measured. The concentration of the particular species, the total amount of the species present in the fermentor, the number of hours the fermentation has been running, and the volume of the fermentor can be considered. In various embodiments, these measurements can be compared to each other and/or they can be compared to previous measurements of the same parameter previously taken from the same fermentation or another fermentation. Adjustments to the amount of a medium component can be accomplished such as by changing the flow rate of a stream containing that component or by changing the frequency of the additions for that component. For example, the amount of saccharide can be increased when the cell production increases faster than the end product production. In another embodiment, the amount of nitrogen can be increased when the enzyme activity level decreases.

In another embodiment, a fed batch operation can be employed, wherein medium components and/or fresh cells are added during the fermentation without removal of a portion of the broth for harvest prior to the end of the fermentation. In one embodiment, a fed-batch process is based on feeding a growth limiting nutrient medium to a culture of microorganisms. In one embodiment, the feed medium is highly concentrated to avoid dilution of the bioreactor. In another embodiment, the controlled addition of the nutrient directly affects the growth rate of the culture and avoids overflow metabolism such as the formation of side metabolites. In one embodiment, the growth limiting nutrient is a nitrogen source or a saccharide source.

In various embodiments, particular medium components can have beneficial effects on the performance of the fermentation, such as increasing the titer of desired products, or increasing the rate that the desired products are produced. Specific compounds can be supplied as a specific, pure ingredient, such as a particular amino acid, or it can be supplied as a component of a more complex ingredient, such as using a microbial, plant or animal product as a medium ingredient to provide a particular amino acid, promoter, cofactor, or other beneficial compound. In some cases, the particular compound supplied in the medium ingredient can be combined with other compounds by the organism resulting in a fermentation-beneficial compound. One example of this situation would be where a medium ingredient provides a specific amino acid which the organism uses to make an enzyme beneficial to the fermentation. Other examples can include medium components that are used to generate growth or product promoters, etc. In such cases, it can be possible to obtain a fermentation-beneficial result by supplementing the enzyme, promoter, growth factor, etc. or by adding the precursor. In some situations, the specific mechanism whereby the medium component benefits the fermentation is not known, only that a beneficial result is achieved.

In one embodiment, a fermentation to produce a fuel is performed by culturing a strain of R. opacus in a medium having a supplement of lignin component and a concentration of one or more carbon sources. The resulting production of end product such as TAG can be up to 1-fold, 2-fold, 3-fold, 4-fold, 5-fold, 6-fold, 7-fold, 8-fold, 9-fold, and in some cases up to 10-fold and higher in volumetric productivity than a process using only the addition of a relatively pure saccharide source, and can achieve a carbon conversion efficiency approaching the theoretical maximum. The theoretical maximum can vary with the substrate and product. For example, the generally accepted maximum efficiency for conversion of glucose to ethanol is 0.51 g ethanol/g glucose. In one embodiment, a biocatalyst can produce about 40-100% of a theoretical maximum yield of ethanol. In another embodiment, a biocatalyst can produce up to about 40%, 50%, 60%, 70%, 80%, 90%, 95% and even 100% of the theoretical maximum yield of ethanol. In one embodiment, a biocatalyst can produce up to about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.99%, or 100% of a theoretical maximum yield of a fuel. It can be possible to obtain a fermentation-beneficial result by supplementing the medium with a pretreatment or hydrolysis component. In some situations, the specific mechanism whereby the medium component benefits the fermentation is not known, only that a beneficial result is achieved.

Various embodiments offer benefits relating to improving the titer and/or productivity of fermentation end-product production by a biocatalyst by culturing the organism in a medium comprising one or more compounds comprising particular fatty acid moieties and/or culturing the organism under conditions of controlled pH.

In one embodiment, the pH of the medium is controlled at less than about pH 7.2 for at least a portion of the fermentation. In one embodiment, the pH is controlled within a range of about pH 3.0 to about 7.1 or about pH 4.5 to about 7.1, or about pH 5.0 to about 6.3, or about pH 5.5 to about 6.3, or about pH 6.0 to about 6.5, or about pH 5.5 to about 6.9 or about pH 6.2 to about 6.7. The pH can be controlled by the addition of a pH modifier. In one embodiment, a pH modifier is an acid, a base, a buffer, or a material that reacts with other materials present to serve to raise of lower the pH. In one embodiment, more than one pH modifier can be used, such as more than one acid, more than one base, one or more acid with one or more bases, one or more acids with one or more buffers, one or more bases with one or more buffers, or one or more acids with one or more bases with one or more buffers. When more than one pH modifiers are utilized, they can be added at the same time or at different times. In one embodiment, one or more acids and one or more bases can be combined, resulting in a buffer. In one embodiment, media components, such as a carbon source or a nitrogen source can also serve as a pH modifier; suitable media components include those with high or low pH or those with buffering capacity. Exemplary media components include acid- or base-hydrolyzed plant polysaccharides having with residual acid or base, AFEX treated plant material with residual ammonia, lactic acid, corn steep solids or liquor.

In one embodiment, a constant pH can be utilized throughout the fermentation. In one embodiment, the timing and/or amount of pH reduction can be related to the growth conditions of the cells, such as in relation to the cell count, the end product produced, the end product present, or the rate of end product production. In one embodiment, the pH reduction can be made in relation to physical or chemical properties of the fermentation, such as viscosity, medium composition, gas production, off gas composition, etc.

Recovery of Fermentation End Products

In another aspect, methods are provided for the recovery of the fermentive end products, such as an alcohol (e.g. ethanol, propanol, methanol, butanol, etc.) another biofuel or chemical product. In one embodiment, broth will be harvested at some point during of the fermentation, and fermentive end product or products will be recovered. The broth with end product to be recovered will include both end product and impurities. The impurities include materials such as water, cell bodies, cellular debris, excess carbon substrate, excess nitrogen substrate, other remaining nutrients, other metabolites, and other medium components or digested medium components. During the course of processing the broth, the broth can be heated and/or reacted with various reagents, resulting in additional impurities in the broth.

In one embodiment, the processing steps to recover end product frequently includes several separation steps, including, for example, distillation of a high concentration alcohol material from a less pure alcohol-containing material. In one embodiment, the high concentration alcoholl material can be further concentrated to achieve very high concentration alcohol, such as 98% or 99% or 99.5% (wt.) or even higher. Other separation steps, such as filtration, centrifugation, extraction, adsorption, etc. can also be a part of some recovery processes for alcohol as a product or biofuel, or other biofuels or chemical products.

In one embodiment, a process can be scaled to produce commercially useful biofuels.

In another embodiment, biocatalyst is used to produce an alcohol, e.g., ethanol, butanol, propanol, methanol, or a fuel such as hydrocarbons hydrogen, TAG, and hydroxy compounds. In another embodiment, biocatalyst is used to produce a carbonyl compound such as an aldehyde or ketone (e.g. acetone, formaldehyde, 1-propanal, etc.), an organic acid, a derivative of an organic acid such as an ester (e.g. wax ester, glyceride, etc.), 1,2-propanediol, 1,3-propanediol, lactic acid, formic acid, acetic acid, succinic acid, pyruvic acid, or an enzyme such as a cellulase, polysaccharase, lipases, protease, ligninase, and hemicellulase.

TAG biosynthesis is widely distributed in nature and the occurrence of TAG as reserve compounds is widespread among plants, animals, yeast and fungi. In contrast, however, TAGs have not been regarded as common storage compounds in bacteria. Biosynthesis and accumulation of TAGs have been described only for a few bacteria belonging to the actinomycetes group, such as genera of Streptomyces, Nocardia, Rhodococcus, Mycobacterium, Dietzia and Gordonia, and, to a minor extent, also in a few other bacteria, such as Acinetobacter baylyi and Alcanivorax borkumensis. Since the mid-1990's, TAG production in hydrocarbon-degrading strains of those genera has been frequently reported. TAGs are stored in spherical lipid bodies as intracellular inclusions, with the amounts depending on the respective species, cultural conditions and growth phase. Commonly, the important factor for the production of TAGs is the amount of nitrogen that is supplied to the culture medium. The excess carbon, which is available to the culture after nitrogen exhaustion, continues to be assimilated by the cells and, by virtue of oleaginous bacteria possessing the requisite enzymes, is converted directly into lipid. The compositions and structures of bacterial TAG molecules vary considerably depending on the bacterium and on the cultural conditions, especially the carbon sources. See, Brigham C J, et al. (2011) J Microbial Biochem Technol S3:002.

In one embodiment, useful biochemicals can be produced from non-food plant biomass, with a steam or hot-water extraction technique that is carried out by contacting a charge of non-food plant pretreated biomass material such as corn stover or sorhum with water and/or acid (with or without additional process enhancing compounds or materials), in a pressurized vessel at an elevated temperature up to about 160-220° C. and at a pH below about 7.0, to yield an aqueous (extract solution) mixture of useful sugars including long-chain saccharides (sugars), acetic acid, and lignin, while leaving the structural (cellulose and lignin) portion of the lignocellulosic material largely intact. In combination, these potential inhibitory chemicals especially sugar degradation products are low, and the plant derived nutrients that are naturally occurring lignocellulosic-based components are also recovered that are beneficial to a C5 and C6 fermenting organism. Toward this objective, the aqueous extract is concentrated (by centrifugation, filtration, solvent extraction, flocculation, evaporation), by producing a concentrated sugar stream, apart from the other hemicellulose (C5 rich) and cellulosic derived sugars (C6 rich) which are channeled into a fermentable stream.

In another embodiment, following enzyme/acid hydrolysis, additional chemical compounds that are released are recovered with the sugar stream resulting in a short-chain sugar solution containing xylose, mannose, arabinose, rhamnose, galactose, and glucose (5 and 6-carbon sugars). The sugar stream, now significantly rich in C5 and C6 substances can be converted by microbial fermentation or chemical catalysis into such products as triacylglycerol or TAG and further refined to produce stream rich in JP8 or jet fuels. If C5 sugar percentage correction has not been performed, it can be performed before fermentation to satisfy desired combination of C5 and C6 sugars for the fermentation organism and corresponding end product.

Biofuel Plant and Process of Producing Biofuel:

Large Scale Fuel and Chemical Production from Biomass Generally, there are several basic approaches to producing fuels and chemical end-products from biomass on a large scale utilizing of microbial cells. In the one method, one first pretreats and hydrolyzes a biomass material that includes high molecular weight carbohydrates to lower molecular weight carbohydrates, and then ferments the lower molecular weight carbohydrates utilizing of microbial cells to produce fuel or other products. In the second method, one treats the biomass material itself using mechanical, chemical and/or enzymatic methods. In all methods, depending on the type of biomass and its physical manifestation, one of the processes can comprise a milling of the carbonaceous material, via wet or dry milling, to reduce the material in size and increase the surface to volume ratio (physical modification).

In one embodiment, hydrolysis can be accomplished using acids, e.g., Bronsted acids (e.g., sulfuric or hydrochloric acid), bases, e.g., sodium hydroxide, hydrothermal processes, ammonia fiber explosion processes (“AFEX”), lime processes, enzymes, or combination of these. Hydrogen, and other end products of the fermentation can be captured and purified if desired, or disposed of, e.g., by burning. For example, the hydrogen gas can be flared, or used as an energy source in the process, e.g., to drive a steam boiler, e.g., by burning. Hydrolysis and/or steam treatment of the biomass can, e.g., increase porosity and/or surface area of the biomass, often leaving the cellulosic materials more exposed to the biocatalyst cells, which can increase fermentation rate and yield. Removal of lignin can, e.g., provide a combustible fuel for driving a boiler, and can also, e.g., increase porosity and/or surface area of the biomass, often increasing fermentation rate and yield. Generally, in any of the these embodiments, the initial concentration of the carbohydrates in the medium is greater than 20 mM, e.g., greater than 30 mM, 50 mM, 75 mM, 100 mM, 150 mM, 200 mM, or even greater than 500 mM.

Biomass Processing Plant and Process of Producing Products from Biomass

In one aspect, a fuel or chemical plant that includes a pretreatment unit to prepare biomass for improved exposure and biopolymer separation, a hydrolysis unit configured to hydrolyze a biomass material that includes a high molecular weight carbohydrate, and one or more product recovery system(s) to isolate a product or products and associated by-products and co-products is provided. In another aspect, methods of purifying lower molecular weight carbohydrate from solid byproducts and/or toxic impurities is provided.

In another aspect, methods of making a product or products that include combining biocatalyst cells of a microorganism and a biomass feed in a medium wherein the biomass feed contains lower molecular weight carbohydrates and unseparated solids and/or other liquids from pretreatment and hydrolysis, and fermenting the biomass material under conditions and for a time sufficient to produce a biofuel, chemical product or fermentive end-products, e.g. ethanol, propanol, hydrogen, succinic acid, lignin, terpenoids, and the like as described above, is provided.

In another aspect, products made by any of the processes described herein is also provided herein.

The following is an example of a method for producing chemical products from biomass by first treating biomass with an acid at elevated temperature and pressure in a hydrolysis unit. The biomass may first be heated by addition of hot water or steam. The biomass may be acidified by bubbling gaseous sulfur dioxide through the biomass that is suspended in water, or by adding a strong acid, e.g., sulfuric, hydrochloric, or nitric acid with or without preheating/presteaming/water addition. During the acidification, the pH is maintained at a low level, e.g., below about 5. The temperature and pressure may be elevated after acid addition. In addition to the acid already in the acidification unit, optionally, a metal salt such as ferrous sulfate, ferric sulfate, ferric chloride, aluminum sulfate, aluminum chloride, magnesium sulfate, or mixtures of these can be added to aid in the acid hydrolysis of the biomass. The acid-impregnated biomass is fed into the hydrolysis section of the pretreatment unit Steam is injected into the hydrolysis portion of the pretreatment unit to directly contact and heat the biomass to the desired temperature. The temperature of the biomass after steam addition is, e.g., between about 130° C. and 220° C. The acid hydrolysate is then discharged into the flash tank portion of the pretreatment unit, and is held in the tank for a period of time to further hydrolyze the biomass, e.g., into oligosaccharides and monomeric sugars. Other methods can also be used to further break down biomass. Alternatively, the biomass can be subject to discharge through a pressure lock for any high-pressure pretreatment process. Hydrolysate is then discharged from the pretreatment reactor, with or without the addition of water, e.g., at solids concentrations between about 10% and 60%.

After pretreatment, the biomass may be dewatered and/or washed with a quantity of water, e.g. by squeezing or by centrifugation, or by filtration using, e.g. a countercurrent extractor, wash press, filter press, pressure filter, a screw conveyor extractor, or a vacuum belt extractor to remove acidified fluid. Wash fluids can be collected to concentrate the C5 saccharides in the wash stream. The acidified fluid, with or without further treatment, e.g. addition of alkali (e.g. lime) and or ammonia (e.g. ammonium phosphate), can be re-used, e.g., in the acidification portion of the pretreatment unit, or added to the fermentation, or collected for other use/treatment. Products may be derived from treatment of the acidified fluid, e.g., gypsum or ammonium phosphate. Enzymes or a mixture of enzymes can be added during pretreatment to hydrolyze, e.g. endoglucanases, exoglucanases, cellobiohydrolases (CBH), beta-glucosidases, glycoside hydrolases, glycosyltransferases, alphyamylases, chitinases, pectinases, lyases, and esterases active against components of cellulose, hemicelluloses, pectin, and starch, in the hydrolysis of high molecular weight components.

A fermentor, attached or at a separate site, can be fed with hydrolyzed biomass, any liquid fraction from biomass pretreatment, an active seed culture of a biocatalyst, such as a yeast, if desired a co-fermenting microbe, e.g., another yeast or E. coli, and, if required, nutrients to promote growth of the biocatalyst or other microbes. Alternatively, the pretreated biomass or liquid fraction can be split into multiple fermentors, each containing a different strain of a biocatalyst and/or other microbes, and each operating under specific physical conditions. Fermentation is allowed to proceed for a period of time, e.g., between about 1 and 150 hours, while maintaining a temperature of, e.g., between about 25° C. and 50° C. Gas produced during the fermentation is swept from fermentor and is discharged, collected, or flared with or without additional processing, e.g. hydrogen gas may be collected and used as a power source or purified as a co-product.

In another aspect, methods of making a fuel or fuels that include combining one or more biocatalyst and a lignocellulosic material (and/or other biomass material) in a medium, adding a lignin fraction from pretreatment, and fermenting the lignocellulosic material under conditions and for a time sufficient to produce a fuel or fuels, e.g., ethanol, propanol and/or hydrogen or another chemical compound is provided herein.

In another aspect, the products made by any of the processes described herein is provided.

### EXAMPLES

The following examples serve to illustrate certain embodiments and aspects and are not to be construed as limiting the scope thereof.

### Example 1

### Procedure for Preserving Ensiled Biomass without the use of Lactobacillus or Other Microbes for Creating an Anaerobic Environment

In this example, biomass will be preserved by an ensiling process that utilizes the addition of one or more gasses and/or one or more acids in order to reduce the oxygen content and acidify the ensiled biomass. The biomass will first be reduced in size and then packed in an air tight container. During the packing process, the one or more gasses and/or one or more acids will be mixed with the biomass particles. The one or more gasses and/or one or more acids will be added in order to reduce the pH of the ensiled biomass to a pH between 4 and 6, endpoints inclusive.

### Example 2

### Comparison of Monomeric Sugar Yields from Silage Produced with Sulfur Dioxide and Silage Produced Through Fermentation Processes

In this example, silage from two different ensiling processes will be pretreated and hydrolyzed to monomeric sugars, and the yields compared. The first ensiling process will be as describe in Example 1 wherein sulfur dioxide gas is used to reduce oxygen levels and acidify the biomass to a pH of between about 4 and 6, endpoints inclusive. The second ensiling process will utilize the fermentation of plant sugars by endogenous microorganisms in the ensiled biomass to reduce oxygen levels and acidify the ensiled biomass. The silage will be pretreated and hydrolyzed in the same manner, which will not include a washing step.

### Example 3

### Comparison of Monomeric Sugar Yields from Silage Produced with Formic Acid and Silage Produced Through Fermentation Processes

In this example, silage from two different ensiling processes will be pretreated and hydrolyzed to monomeric sugars, and the yields compared. The first ensiling process will be as describe in Example 1 wherein formic acid is used to reduce oxygen levels and acidify the biomass to a pH of between about 4 and 6, endpoints inclusive. The second ensiling process will utilize the fermentation of plant sugars by endogenous microorganisms in the ensiled biomass to reduce oxygen levels and acidify the ensiled biomass. The silage will be pretreated and hydrolyzed in the same manner, which will not include a washing step.

### Example 4

### Comparison of Monomeric Sugar Yields from Silage Produced with Formic Acid and Sulfur Dioxide and Silage Produced Through Fermentation Processes

In this example, silage from two different ensiling processes will be pretreated and hydrolyzed to monomeric sugars, and the yields compared. The first ensiling process will be as describe in Example 1 wherein formic acid and sulfur dioxide are used to reduce oxygen levels and acidify the biomass to a pH of between about 4 and 6, endpoints inclusive. The second ensiling process will utilize the fermentation of plant sugars by endogenous microorganisms in the ensiled biomass to reduce oxygen levels and acidify the ensiled biomass. The silage will be pretreated and hydrolyzed in the same manner, which will not include a washing step.

### Example 5

### Comparison of Monomeric Sugar Yields from Silage Produced with Propionic Acid and Silage Produced Through Fermentation Processes

In this example, silage from two different ensiling processes will be pretreated and hydrolyzed to monomeric sugars, and the yields compared. The first ensiling process will be as describe in Example 1 wherein propionic acid is used to reduce oxygen levels and acidify the biomass to a pH of between about 4 and 6, endpoints inclusive. The second ensiling process will utilize the fermentation of plant sugars by endogenous microorganisms in the ensiled biomass to reduce oxygen levels and acidify the ensiled biomass. The silage will be pretreated and hydrolyzed in the same manner, which will not include a washing step.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention. It is intended that the following claims define the scope of the invention and that methods and structures within the scope of these claims and their equivalents be covered thereby.

1. A method of producing silage from a biomass, the method comprising:
(a) packing the biomass in a container to a bulk density sufficient to reduce oxygen levels within and surrounding the biomass;
(b) contacting the biomass with sulfur dioxide gas, formic acid, propionic acid, or a combination thereof to acidify the biomass; and
(c) maintaining the biomass under conditions and for a time to produce silage from the biomass.

2. The method of claim 1, further comprising
(c)
(d) pretreating or hydrolyzing the silage to produce one or more sugar streams.

3. The method of claim 2, wherein the silage does not require washing prior to pretreating or hydrolyzing.

4. The method of claim 1, wherein less lactic acid is produced compared to an ensiling process that does not include contacting a biomass with sulfur dioxide gas, formic acid, propionic acid, or a combination thereof.

5. The method of claim 1, wherein no lactic acid is produced.

6. The method of claim 2, comprising pretreating the silage, wherein pretreating comprises dilute acid hydrolysis, wherein less acid is required during pretreatment than for silage produced by an ensiling process that does not include contacting a biomass with sulfur dioxide gas, formic acid, propionic acid, or a combination thereof.

7. The method of claim 2, wherein the one or more sugar streams comprise a higher yield of monomeric sugars in comparison to a yield from silage produced by an ensiling process that does not include contacting a biomass with sulfur dioxide gas, formic acid, propionic acid, or a combination thereof.

8. The method of claim 1, wherein the silage contains a lower level of bacteria, mold, and/or yeast than a silage produced by an ensiling process that does not include contacting a biomass with sulfur dioxide gas, formic acid, propionic acid, or a combination thereof.

9. The method of claim 1, wherein the biomass has a pH of from about 4 to about 6 after contacting the biomass with sulfur dioxide gas, formic acid, propionic acid, or a combination thereof.

10. The method of claim 1, comprising contacting the biomass with the formic acid, wherein the formic acid is in an amount of from about 0.1% to about 20% of the biomass by dry matter weight.

11. The method of claim 1 or 2, comprising contacting the biomass with the formic acid, wherein the formic acid is in an amount of from about 0.1% to about 5% of the biomass by dry matter weight.

12. The method of claim 1, comprising contacting the biomass with the sulfur dioxide gas, wherein the sulfur dioxide gas is in an amount of from about 0.1% to about 20% of the biomass by dry matter weight.

13. The method of claim 1, comprising contacting the biomass with the sulfur dioxide gas, wherein the sulfur dioxide gas is in an amount of from about 0.1% to about 5% of the biomass by dry matter weight.

14. The method of claim 1, comprising contacting the biomass with the proprionic acid, wherein the propionic acid is in an amount of from about 0.1% to about 20% of the biomass by wet matter weight.

15. The method of claim 1, comprising contacting the biomass with the proprionic acid, wherein the propionic acid is in an amount of from about 0.1% to about 5% of the biomass by wet matter weight.

16. The method of claim 1, further comprising contacting the biomass with carbon dioxide to reduce oxygen levels.

17. (canceled)

18. The method of claim 1, wherein the biomass is reduced to an average size of from about 0.6 to about 1.3 cm prior to packing the biomass in the container.

19. The method of claim 1, wherein the bulk density is from about 10 lbs/ft³ to about 100 lbs/ft³.

20. (canceled)

21. (canceled)

22. (canceled)

23. The method of claim 1, wherein the biomass has a moisture content of from about 50% to about 70%.

24. (canceled)

25. The method of claim 1, wherein the container is an air-tight container.

26. (canceled)

27. (canceled)

28. The method of claim 1, wherein the silage comprises starch and the method further comprises contacting the biomass with an amylase.

29. (canceled)

30. The method of claim 1, wherein the biomass comprises crops or crop residues from grasses, herbaceous legumes, tree legumes, corns or maize, alfalfas, sorghums or sweet sorghums, oats, rice, wheat, barley, millets, triticale, ryes, buckwheat, fonios, quinoa, or a combination thereof.

31.-55. (canceled)
